(19) United States
(12) Patent Application Publication
(10) Pub. No.: US 2021/0230248 A1
(43) Pub. Date: Jul. 29, 2021

(54) Multivalent pharmacophores for high avidity and overexpressed-target specific binding and uses thereof

(71) Applicant: Jun Chen, (US)

(72) Inventor: Jun Chen, (US)

(21) Appl. No.: 16/752,644

(22) Filed: Jan. 25, 2020

(51) Int. Cl. C07K 14/705

(52) U.S. Cl. CPC C07K 14/70521; A61K 38/00; C07K 14/70596

(57) ABSTRACT

Overexpression of a variety of cell surface markers in cancer cells and/or non-cancer cells in the tumor microenvironment is an important hallmark for many types of cancers and is associated with cancer progression and poor prognosis. This invention provides multivalent pharmacophores for high avidity and specific binding to these overexpressed markers with much reduced binding to normally-expressed targets in healthy tissues. Further, the pharmacophores will not be interfered by soluble targets present in the circulatory systems and in tumor microenvironment. This new class of targeting therapeutics and diagnostics will provide better efficacy in cancer treatment and higher accuracy in cancer diagnosis than the currently available therapeutic and diagnostic means. This invention will also expand the range of targets that can be targeted in both cancer treatment and diagnosis, and more types of cancers can be treated target-specifically. Other diseases that have overexpressed cell surface markers in diseased cells will also be benefitted from this invention.

### BACKGROUND OF THE INVENTION

One of the most prominent features of cancer is overexpression of some cell surface proteins and non-protein cell membrane components such as glycolipids in cancer cells and/or non-cancer cells in tumor microenvironment. These overexpressed surface markers include PD-L1, PD-L2, PD-1, B7-H3, B7x, B7-H4, galectins, TIM-3, CD74, CD47, CD24, CXCR4, folate receptor, transferrin receptor (TfR), EGFR, EGFRvIII, HER2, HER3, HER4, PDGFRα and β, FGFRs, ALK, EphA2, insulin-like growth factor receptors (IGF-1R and INSR-A), ATP-binding cassette (ABC) transporters (P-gp, BCRP and MRP1), claudins, EpCAM, carcinoembryonic antigen-related cell adhesion molecules (CEA and CEACAM6), CD44, integrins, urokinase-type plasminogen activator receptor (uPAR), type II transmembrane serine proteases (matriptase, hepsin and TMPRSS2), proteoglycans (CSPG4, glypicans and syndecans), mucins, mesothelin, carbonic anhydrase IX and XII, cancer-testis antigens (MAGEs and NY-ESO-1), and gangliosides (GD2 and GD3). The overexpression of these cell surface markers is important for cancer immune evasion, carcinogenesis, cancer cell proliferation and metastasis, and resistance to apoptosis and therapeutic agents. Many of these markers are closely correlated with poor prognosis. Significantly, they also provide characteristics that differentiate cancer cells and cancer stromal cells from normal cells in healthy tissues, and can be exploited for selective targeting. However, current technologies lack the necessary tools to effectively exploit these differences and new approaches are needed.

The current trend on drug development for cancer targeted therapy is to emphasize high affinity as one of the main focus and development strategies. The higher affinity a drug has, the more efficacious it usually becomes if specificity is not a concern. However, all of the targeted drugs that are either approved for clinical use or still in development exhibit off-target and/or on-target adverse events.

Therapeutic antibody is famous for its specificity. Since FDA's approval of Muromonab-CD3, the first monoclonal antibody approved more than 30 years ago, more than fifty antibodies have been approved. Many of these antibodies are for cancer treatment, and the targets include CD20, CD38, HER2, EGFR, VEGFR, VEGFR2, PDGFRα, IL-1α, mucin1, GD2, CTLA-4, PD-1, and PD-L1. Bi-specific antibodies targeting CD19 and CD3 or EpCAM and CD3 are also in clinic use. However, all of these targets are not only expressed in cancer cells and/or non-cancer cells including immune cells in the tumor microenvironment, but also in normal cells of healthy tissues. Therefore, the on-target/off-tumor adverse events in various severities are observed in patients with the associated treatment. For example, cetuximab and panitumumab, a chimeric monoclonal antibody and a fully human monoclonal antibody, respectively, target EGFR and increase progression-free and overall survival in wide-type KRAS colorectal cancer patients. However, EGFR is constitutively expressed in many normal epithelial tissues, and patients receiving anti-EGFR antibody commonly show symptoms of skin toxicity and an increased risk of diarrhea and mucositis (Miroddi et al., Crit Rev Oncol Hematol, 2015, 96(2):355-71; Hofheinz et al., Crit Rev Oncol Hematol, 2017, 114:102-13). Some of these side effects are severe and life-threatening. Trastuzumab is a monoclonal antibody targeting HER2 and used in HER2 overexpressing breast cancer. Cardiac toxicity is a major side effect of trastuzumab, with a chronic progressive deterioration of left ventricular ejection fraction, up to congestive heart failure (Procter et al., J Clin Oncol, 2010, 28(21):3422-8; Meattini et al., Med Oncol, 2017, 34(5):75).

Cancer immunotherapy is coming of age. It has prompted a paradigm shift in oncology, in which therapeutic agents are used to target immune cells rather than cancer cells. The first generation of cancer immunotherapy is antagonistic antibodies to immune checkpoint molecules, such as cytotoxic T-lymphocyte-associated antigen 4 (CTLA-4), programmed cell death protein-1 (PD-1) and its ligand PD-L1. Targeting these checkpoints has led to long-lasting tumor responses, yet side effects are also observed. Although antibodies to PD-1 and PD-L1 are generally considered well tolerated compared to anti-CTLA-4 antibody, immune-related adverse events (irAE) are still very common (˜70%) and occur across different tumor types, with some of them requiring hospitalization and even life-threatening (Sosa et al., Ther Adv Med Oncol, 2018, 10:1758834018764628; Callahan et al., Immunity, 2016, 44(5):1069-78). Of note, the combined treatment with two checkpoint inhibitors, ipilimumab (anti-CTLA-4) and nivolumab (anti-PD-1), has resulted in substantially increased irAE compared with single checkpoint inhibitor. In melanoma, adverse events related to the combination were reported in 96% patients, with up to 59% (Wolchok et al., N Engl J Med, 2017, 377(14):1345-56) or 54.9% (D'Angelo et al., J Clin Oncol, 2017, 35(2):226-35) of grade 3-4 adverse events. In a Phase I study of the combination in renal cell carcinoma, 71.3% patients experienced a grade 3 or 4 irAE and 50.0% experience treatment-related irAE classified as grade 3 or 4 (Hammers et al., J Clin Oncol, 2017, 35(34):3851-8). Although any system in the body can be affected by irAE, the predominantly involved organs include skin, gastro-intestine, liver, lungs and endocrine glands. Because immune checkpoints play pivotal roles in the maintenance of self-tolerance, a non-discriminated blockade can alter immunological tolerance and give rise to autoimmune or inflammatory side effects. It is reported that approximately 13% of patients with lung cancer have one or more autoimmune diseases at any time and most clinical trials exclude patients with autoimmune diseases (Khan et al., Medicine (Baltimore), 2018, 97(33):e11936). It will be a challenge to treat this group of patients with immune checkpoint inhibitors. In addition, PD-1 and PD-L1 are constitutively expressed at low levels by cardiomyocytes, and a number of cardiotoxic events (myocarditis, cardiac failure, heart block, myocardial fibrosis and cardiomyopathy) were documented in patients treated with checkpoint inhibitors (Varricchi et al., ESMO Open, 2017, 2(4):e000247).

Despite their relatively high selectivity, therapeutic antibodies can have non-specific interactions, leading to off-target binding and toxicity as well as fast clearance in vivo (Starr et al., Curr Opin Biotechnol, 2019, 60:119-27). Non-specific antibody interactions are generally thought to be driven, in large part, by electrostatic and hydrophobic interactions. What makes the issue on antibody specificity complicated is some of the same residues that contribute to off-target binding also promote strong interaction with the target antigen. Another cause of antibody's non-specific binding is through Fc receptors on the surface of immune cells.

Besides specificity related issues, another antibody inherent limitation is its large size with the molecular weight at about 150 Kd, which can curtail the efficacy of antibodies due to limited tissue/tumor penetration.

Nature makes extensive use of multivalent interactions, which involves the simultaneous binding of multiple ligands on one biological entity to multiple receptors on another. The multivalent interaction can be much stronger than the corresponding monovalent interactions combined, and display selective behavior toward receptors above a threshold concentration (Dubacheva et al., J Am Chem Soc, 2019, 141(6):2577-88; Chittasupho, Ther Deliv, 2012, 3(10):1171-87). The high avidity or functional affinity associated with multivalent interaction comes from the mechanism that binding of one ligand of a multivalent entity to its target causes the unbound but tethered ligands to stay in “forced proximity” to the nearby targets, and thus forms a local high concentration of ligands (FIG. 1). As a result, the unbound ligands are more likely to bind to these targets (Vauquelin and Charlton, Br J Pharmacol, 2013, 168(8):1771-85; Kitov and Bundle, J Am Chem Soc, 2003, 125(52):16271-84; Bobrovnik, J Mol Recognit, 2007, 20(4):253-62). The multivalent binding also has longer residence time to their targets because of the effect of hindered ligand diffusion, which results in increased probability of rebinding when freshly dissociated.

The avidity of multivalent interaction is determined not only by the affinity of individual ligand, but more importantly by the ligands' effective concentration. The effective concentration is described as a concentration of ligands that can effectively interact with the cognate receptors in the local area. If the effective concentration is high, the probability of interaction between ligands and receptors in the local area is also high. Effective concentration is determined by several factors, including the valency, the structural feature of the scaffold to which the ligands are linked, the linker length (or the distance between the ligands), and the degree of freedom of coupling between the ligands and the cognate receptors.

The valency of a multivalent entity is defined as the number of binding ligands on the entity. In general, the increase of valency increases effective concentration and dramatically improves the avidity. The structural feature of scaffold, including the size and architecture of the scaffold, determines how the ligands are arranged, and the orientation and the density of ligands. For example, the ligands on a branched-chain or star-shaped scaffold will be arranged more closely together than on a linear chain, and have higher effective concentration and less variable distance between ligands (FIG. 2). Reducing linker length usually increases the density of the ligands and thus effective concentration (Shewmake et al., Biomacromolecules, 2008, 9(11):3059-64). However, the linker length needs to be adjusted by the density of cognate receptors. For example, a longer linker is needed for the receptors with low density in order for the linked ligands to reach multiple receptors simultaneously; otherwise the effective concentration will approach zero. The linker also needs to be long enough to allow the linked ligands to orient themselves freely for effective interaction with the receptors. The degree of freedom of coupling between the linked ligands and the receptors has a significant effect on avidity. Rigid linkers and scaffold with limited flexibility would restrict ligands' orientation and effective interactions with cognate receptors. Flexible multivalent structure will allow ligands to adopt a variety of conformations and orientations to effectively bind the targets with low steric strains.

Another important feature of multivalent interaction is that the successful multivalent binding and the associated high avidity also depend on the density of targets. If the distance between targets is longer than that between ligands, the multivalent interaction will not occur. Consequently, by adjusting the distance between ligands, a multivalent entity can discriminate between different target densities (Zuckier et al., Cancer Res, 2000, 60(24):7008-13; Muller et al., Anal Biochem, 1998, 261(2):149-58). Since overexpression of many cell surface markers is common in cancer cells and/or non-cancer cells in the tumor microenvironment, a multivalent entity can selectively bind to these overexpressed targets with high avidity, but weakly to non-overexpressed targets. Further, if a multivalent entity is composed of ligands with low to moderate affinity or no higher affinity than the endogenous ligands, a monovalent binding between the multivalent entity and one of its targets will not happen, and as such the binding of the entity to normally-expressed targets in the healthy tissue can largely be avoided. Therefore, a well designed multivalent entity can achieve the binding that is highly selective and has strong avidity to the overexpressed targets in tumors without or with much reduced side effects associated with on-target/off-tumor interactions. Moreover, because the multivalent entity selects and binds to overexpressed targets and has no Fc-related binding, the off-target binding will also be less likely.

Cell surface protein shedding, in which the extracellular portion of the surface protein is cleaved and released from the cell membrane, happens to many cell surface proteins especially to those overexpressed in cancer cells. Some of the examples are PD-1, PD-L1, B7-H3, galectins, TIM-3, CD74, EGFR, HER2, FGFRs, EphA2, EpCAM, CEA, CD44, uPAR, matriptase, CSPG4, glypican-3, syndecan-1, mucins, mesothelin, and carbonic anhydrase IX. High concentrations of the cleaved and soluble cell surface proteins/peptides present in the blood and lymphatic circulatory systems and in tumor microenvironment can compete for binding to high affinity therapeutic agents, such as antibodies, and limit the access of these agents to the targets on the cell membrane. Moreover, the binding of antibodies to the soluble targets can cause rapid clearance of the antibodies from the body. Additionally, binding of antibody to its soluble target forms an immune complex which can potentially cause immune complex diseases when deposited in organs (Rojko et al., Toxicol Pathol, 2014, 42(4):725-64; Theofilopoulos and Dixon, Am J Pathol, 1980, 100(2):529-94). This specific type of on-target/off-tumor interaction can be avoided by a multivalent entity if the ligands of the entity have low to moderate affinity and a monovalent binding does not occur.

Collectively, a well-designed multivalent pharmacophores as described in this invention can accomplish high avidity and selectivity on both on-target/off-tumor and off-target interactions when targeting overexpressed cell surface targets. In addition, a multivalent pharmacophore can have smaller molecular weight than antibodies and thus better tissue penetration. Furthermore, by clustering the cell surface markers, a multivalent pharmacophore has the potential to induce or inhibit endocytosis of the targets and thus to affect cell growth or death (Daniels et al., Clin Immunol, 2006, 121(2):144-58), or to disrupt the functions of the bound markers. All these characteristics and advantages possessed by multivalent interactions, which the high affinity targeting therapeutics such as antibody do not have, make multivalent pharmacophore a different class of targeted therapy that can provide better treatment to cancer patients than what is possible with the currently available targeted therapy. Although a lot of cell surface markers have been found overexpressed in a variety of cancers, current targeted therapy has only successfully targeted a small fraction of them partly due to lack of selectivity between cancers and healthy tissues. As a different class of targeted therapy, multivalent pharmacophore will be able to target many more of these cell surface markers and many types of cancers can be treated target-specifically as a result.

It is well understood that cell surface targets are not static on the cell membrane and the effect of lateral mobility and target clustering can complicate the multivalent interactions. However, the selective binding behavior of multivalent probes has been demonstrated in biological membranes and proven to be similar to the immobile target binding (Dubacheva et al., J Am Chem Soc, 2019, 141(6):2577-88).

U.S. Pat. No. 8,216,996 B2 (2012), US patent application US2012/0269859 A1, and U.S. Pat. No. 8,574,872 B2 (2013), all titled “Multimer of extracellular domain of cell surface functional molecule”, describe a multimer composed of an extracellular domain of a cell surface functional molecule, particularly a tetramer of an extracellular domain of PD-1 or PD-L1, for the treatment of cancer and other diseases. However, there are major differences between the present invention and the patents: (1) The purpose of U.S. Pat. No. 8,216,996, US2012/0269859 and U.S. Pat. No. 8,574,872 is to develop an alternative substance to antibody, in order to avoid advanced production technology and facilities for formulation associated with antibody production. Contrarily, the present invention pursues a substance that targets overexpressed cell surface markers and has better efficacy than antibody or other similar products. (2) Unlike present invention, U.S. Pat. No. 8,216,996, US2012/0269859 and U.S. Pat. No. 8,574,872 have no intention to specifically target the overexpressed cell surface markers. (3) The multimer described in U.S. Pat. No. 8,216,996, US2012/0269859 and U.S. Pat. No. 8,574,872 is composed of extracellular domains of PD-1 or PD-L1 that are serially-concatenated directly or with linkers. This arrangement of targeting elements in a linear chain usually leads to lower effective concentration of the element (that is, lower avidity) than the architecture described in the present invention in which all the elements are arranged in a form of cluster and are close together. As indicated by Tito and Frenkel (Macromolecules, 2014, 47(21):7496-7509), the targeting elements arranged in a linear chain have less freedom to adopt a variety of conformations and orientations for effective binding, and the binding probability is less on the extremities of a linear chain. Additionally, placing targeting elements uniformly along the chain backbone reduces or eliminates the possibility for cooperative binding. More importantly, linear arrangement of targeting elements is less capable of sharply discriminating between the overexpressed markers and the normally-expressed ones, because the distance between the targeting elements is varied widely (from the shortest as between the two neighboring elements, to the longest as between the two extremities), and the binding valency fluctuates. (4) According to U.S. Pat. No. 8,216,996, US2012/0269859 and U.S. Pat. No. 8,574,872, the extracellular domains can also be linked by avidin, streptavidin or a derivative. Although, with such linkers, the extracellular domains could be arranged in a form of cluster, these linkers have very limited flexibility, which will substantially restrict the freedom of the linked ligands and decrease the avidity. Moreover, avidin and streptavidin are non-human proteins and will induce immunity after repeated use in human. (5) In order to reduce non-specific binding to normally-expressed targets in healthy tissues, the present invention emphasizes that the ligands of a multivalent entity have low to moderate affinities to their cognate sites or no higher affinity than endogenous ligands. However, no such attention is paid in U.S. Pat. No. 8,216,996, US2012/0269859 and U.S. Pat. No. 8,574,872, and instead they include modifying some amino acids in the extracellular domains of the PD-1 or PD-L1 with the purpose of enhancing binding affinity or other properties. (6) The multimer of U.S. Pat. No. 8,216,996, US2012/0269859 and U.S. Pat. No. 8,574,872 can have peptide linkers of 5-15 amino acids. However, no criteria were given on how to select the number of amino acids for the linker, probably because there is no intention for the multimer to be used specifically on overexpressed targets. In contrast, the linker length in the present invention is given between 2 nm to 60 nm, with each specific linker length decided by the density of overexpressed targets and the freedom and accessibility of the linked ligand to the cognate markers.

U.S. Pat. No. 6,511,663 B1 (2003) titled “Tri- an tetra-valent monospecific antigen-binding proteins”, describes a antigen-binding protein comprising three or four Fab fragments bound to each other covalently by a connecting structure for treatment and diagnosis of cancer. The patent is substantially different from the present invention: (1) U.S. Pat. No. 6,511,663 has no intention to specifically target the overexpressed cell surface targets and the described antigen-binding proteins are unlikely to selectively bind to the overexpressed cell surface targets either. (2) In its linker design, U.S. Pat. No. 6,511,663 has no intention to optimize the length of the branches based on the density of cell surface targets in order to have discriminatory binding between overexpressed and normally-expressed targets and maximize the effective concentration of the linked Fab fragments. (3) The linker as designed in U.S. Pat. No. 6,511,663 is not very flexible and therefore the avidity will be reduced. (4) The Fab fragments in U.S. Pat. No. 6,511,663 are not designed to have low to moderate affinity to their antigens in order to limit binding to the targets normally expressed in healthy tissues.

### BRIEF SUMMARY OF THE INVENTION

High affinity targeting therapeutics, such as antibodies and other similar agents, strongly bind to cell surface markers expressed in tumors and have shown substantial efficacy in cancer treatment. However, off-target and, especially, on-target/off-tumor toxicity present a significant challenge for these targeting therapeutics and better technologies are needed.

A variety of cell surface proteins and non-protein surface markers are overexpressed in cancer cells and/or non-cancer cells in the tumor microenvironment, which is associated with the disease progression and treatment resistance. However, there is no effective tool available to selectively target these overexpressed markers, and so far only a small number of them have been targeted successfully in cancer treatment. By applying the principles of multivalency, the multivalent pharmacophores as disclosed in the invention can overcome these obstacles, and provide better efficacy in cancer treatment and higher accuracy in cancer diagnosis than the currently available therapeutic and diagnostic means.

Compared with therapeutic antibodies and other high affitiny targeting therapeutics, the multivalent pharmacophores presented in this invention have these advantages:
1. Overexpressed-target specificity: The multivalent pharmacophores can discriminate between the overexpressed cell surface markers in cancers and normally-expressed ones in healthy tissues;
2. High avidity: The multivalent pharmacophores can obtain higher avidity than the affinity of antibodies toward the overexpressed targets;
3. The pharmacophores will not be interfered by high concentration of cleaved and soluble targets present in the circulatory systems and in tumor microenvironment.
4. By clustering the cell surface markers, the multivalent pharmacophores have the potential to induce or inhibit endocytosis of the bound targets, disrupt their functions and induce cell growth inhibition or death;
5. The multivalent pharmacophores can be synthesized with lower molecular weight than antibodies, thereby having better tissue penetration property;
6. Due to the specificity of the multivalent pharmacophores, many overexpressed cell surface markers can be targeted;
7. Many more types of cancers will be treated target-specifically by the multivalent pharmacophores.

To demonstrate the capabilities of the presently disclosed invention, three cell surface markers are targeted by specific multivalent pharmacophores as examples for cancer treatment.

PD-1-PD-L1/PD-L2 immune checkpoint plays important role in cancer's immune evasion. PD-1 is a receptor for both PD-L1 and PD-L2, and is expressed mainly on the surface of activated T cells, B cells, and monocytes/macrophages. Besides functioning as the ligand for PD-1, PD-L1 also engages CD80 to deliver bidirectional inhibitory signals to activated T cells. PD-L1 and PD-L2 are often overexpressed in cancer cells and cancer stromal cells. The antagonistic antibodies to PD-1 and PD-L1 have achieved remarkable success in treatment of multiple types of cancers. Nevertheless, the response rates in patients are generally low, which is likely due to the complex network of immunosuppressive pathways present in advanced tumors that cannot be overcome by a single checkpoint blockage. At the same time, because of inhibition of immune checkpoint in normal immunity, the treatment causes a variety of immune-related side effects.

Anti-PD-1 antibody blocks the engagement of PD-1 with its ligands, PD-L1 and PD-L2, and anti-PD-L1 antibody blocks interactions between PD-L1 and PD-1 or CD80. Therefore, neither anti-PD-1 nor anti-PD-L1 antibody alone can block all the checkpoint signalings that involve the axes of PD-1/PD-L1, PD-1/PD-L2, and CD80/PD-L1. An ectodomain of PD-1 can interact with both PD-L1 and PD-L2 and prevent their engagement with cell surface bound PD-1. Further, because both PD-1 and CD80 have overlapping binding site on PD-L1, the ectodomain of PD-1 also prohibits interaction between PD-L1 and CD80. Therefore, an ectodomain of PD-1 can have a function equivalent to the combined actions of anti-PD-1 and anti-PD-L1 antibodies. However, the affinity between PD-1 and PD-L1 or PD-L2 is low, with Kd values of ˜8 μM and ˜2 μM, respectively. It is impossible to have any therapeutic effect by administration of PD-1 ectodomain in patients, especially considering the fact that both PD-1 and PD-L1 are usually overexpressed in tumors. A multivalent pharmacophore with PD-1 ectodomain as the ligands can exhibit high avidity and specificity to overexpressed PD-L1 and PD-L2 in tumors. At the same time, side-effects caused by off-target and/or on-target/off-tumor binding will be limited. Especially, the cancer patients with autoimmune diseases can be treated with much fewer risks of toxicity from general immune checkpoint inhibition. Furthermore, the multivalent pharmacophore will not be interfered by high concentrations of cleaved and soluble PD-L1 in the circulatory systems and in tumor microenvironment. As examples, 4-arm PEG PD-1_{ecto} and 6-arm PEG PD-1_{ecto} pharmacophores were synthesized. They showed selective binding to the plate with high PD-L1 density and to PD-L1 overexpressing SU-DHL-1 cells.

Another intensely studied immuno-oncology target is signal-regulatory protein (SIRP)α-CD47 immune checkpoint. SIRPα is expressed on myeloid cells, including macrophages, dendritic cells and neutrophils. CD47 is expressed on virtually all cells, including red blood cells (RBCs) and platelets. CD47 interacts with SIRPα through its Ig-like domain to the N-terminal IgV-like domain of SIRPα, thereby suppressing the activation of myeloid cells. Therefore, binding of CD47 to SIRPα on myeloid cells conveys a ‘don't eat me’ signal. CD47 is overexpressed in numerous hematologic malignancies and solid tumors to evade myeloid cell surveillance. Blockade of CD47-SIRPα interaction can induce phagocytosis of tumor cells. The blockade also promotes development of anti-tumor adaptive T cell responses, possibly as a consequence of increased tumor cell uptake by professional antigen-presenting cells and enhanced antigen cross-presentation.

Given the nearly ubiquitous expression of CD47 at low levels in normal tissues and the homeostatic functions of the CD47-SIRPα interaction, the checkpoint blockade with high affinity therapeutics such as antibody can cause side effects, including anemia, thrombocytopenia and leucopenia. In addition, due to the extensive expression of CD47 especially on RBCs, anti-CD47 agents will face a huge antigen sink and require larger and more frequent drug administration. Owing to its high avidity and specificity to the overexpressed targets, a multivalent pharmacophore can overcome these obstacles. In this invention, multivalent pharmacophores are designed using SIRPα IgV-like domain as ligands for targeting cancer cells with CD47 overexpression. As examples, 4-arm PEG-SIRPα IgV and 6-arm PEG-SIRPα IgV pharmacophores are described in this invention, which are synthesized with both wide-type and Q67R mutant SIRPα IgV-like domain. The pharmacophores exhibited highly competitive binding to CD47 overexpressing Jurkat cells, while avoiding binding to RBCs. An in vitro phagocytosis assay showed that the multivalent PEG-SIRPα IgV pharmacophores promoted phagocytosis of Raji cells by macrophages and enhanced the phagocytic effect of rituximab when combined. The studies suggest that the pharmacophores will offer better and safer therapy than antibody and other similar therapeutics.

Urokinase-type plasminogen activator system is involved in many physiologic and pathologic processes. Urokinase-type plasminogen activator (uPA) binds to its receptor uPAR through its N-terminal growth factor-like domain (GFD) with high affinity (Kd<0.5 nM). uPAR is expressed in many normal cells including neutrophils, T lymphocytes, monocytes-macrophages and fibroblasts, and is overexpressed in a variety of cancers. The overexpression also exhibits a strong correlation with poor cancer prognosis. Binding of uPA to uPAR enhances the efficiency of uPA catalyzed plasminogen activation and is critical to pericellular proteolytic cascade. The binding also initiates various signaling pathways involving cell adhesion and migration. As a result, binding of uPA to uPAR plays important roles in tumor cell adhesion, invasion and metastasis.

Thus, blocking uPA-uPAR interaction has become an attractive therapeutic strategy. However, no uPA-uPAR targeting therapeutic agent has been developed beyond Phase II clinical trial despite over two decades of efforts. Besides the challenge imposed by remarkable species specificity between human and other species for the system, the very high affinity between uPA and uPAR (Kd<0.5 nM), which is similar to that of most antibodies, makes competitive inhibitors difficult to compete in the tumor microenvironment where both uPA and uPAR are usually overexpressed.

In this invention, multivalent pharmacophores are designed by using growth factor-like domain (GFD) of uPA as the ligands. In particular, 4-arm PEG-GFD and 6-arm PEG-GFD are synthesized and tested. Competitive binding assay showed that the multivalent pharmacophores had much stronger avidity than uPA in binding to uPAR overexpressing Hela cells, and could efficiently dislodge uPA already bound to uPAR in HT-1080 cells. Unlike uPAR blocking antibody, the 4-arm and 6-arm pharmacophores could substantially inhibit the adhesion, migration and invasion of cancer cells that overexpress uPAR due to their high avidity.

It is important to point out that what have been described above are just examples a multivalent pharmacophore can accomplish in different situations and demonstrate advantages to currently used targeting therapeutics such as antibodies. Other targets, applications, features, and advantages of the present invention will be apparent to one of skill in the art.

In addition to the therapeutic purpose, the presently disclosed and claimed inventive concepts have potential application for disease prevention, diagnosis, prognosis or any combination of them.

The multivalent pharmacophores described in this invention can not only be used for cancer treatment and diagnosis, but also for treatment and diagnosis of other diseases that show overexpression of cell surface markers in the diseased cells, including chronic viral, bacterial, and parasitic infectious diseases.

### BRIEF DESCRIPTION OF THE DRAWINGS

FIG. 1 describes the mechanism of multivalent binding and the concept of effective concentration. Bivalent entity is used for simplicity. Binding of one ligand of a bivalent entity to its target causes the other unbound but tethered ligand to stay in “forced proximity” to the nearby targets and leads to a local high concentration of ligands around the targets. The multivalent binding also has longer residence time to their targets because of the effect of hindered ligand diffusion, which results in increased probability of rebinding when freshly dissociated. Effective concentration is determined by the valency, the structural feature of the scaffold to which the ligands are linked, the linker length (or the distance between the ligands), and the degree of freedom of coupling between the ligands and the cognate receptors

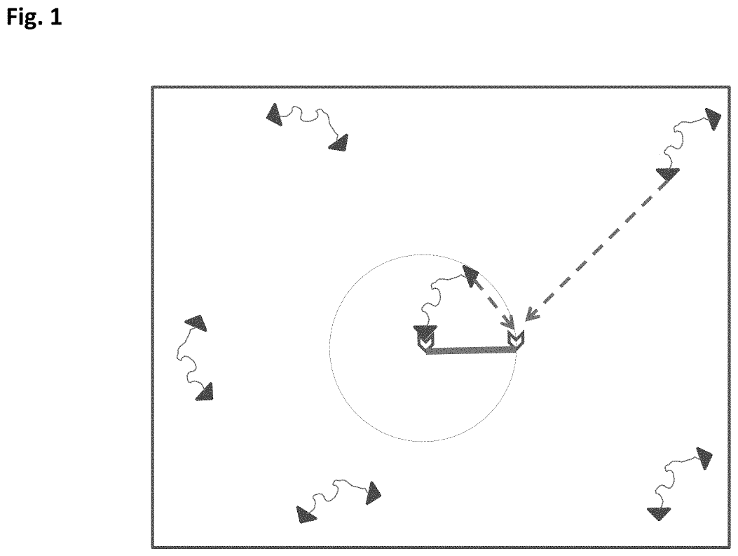

FIG. 2A-2C are descriptive illustrations for different types of linker scaffold. FIG. 2A is a linear linker, FIG. 2B is a branched linker, and FIG. 2C is a star-shaped linker. The filled triangles at the tip of linker branches represent ligands. The ligands in branched linker and star-shaped linker are more close together and have higher effective concentration than those in the linear linker. On the other hand, compared with the branched or star-shaped linker, the distance between ligands in the linear linker is more variable, with the shortest distance between two neighboring ligands and the longest one between the two extremities.

FIG. 3A-3B outline the process of C-terminal site-specific conjugation of the protein of interest (eg. PD-1_{ecto}) to branched PEG. FIG. 3A describes the process for generation of protein of interest (eg. PD-1_{ecto}) C-terminal hydrazide. The protein of interest is genetically fused to the N-terminus of an engineered GyrA intern and chitin binding domain (CBD), expressed in E. coli and purified by immobilization onto chitin beads. An N to S acyl shift at the protein-intein junction forms a branched thioester intermediate which is chemically cleaved using hydrazine to liberate the corresponding C-terminal hydrazide derivative of the protein. FIG. 3B describes the process for generation of pyruvoyl branched PEG and site-specific C-terminal PEGylation of the protein of interest. 4-arm homofunctional PEG amine is used for an example. The 4-arm PEG amine in anhydrous DCM is treated with pyruvoyl chloride under N₂ in the presence of triethylamine at 0° C. overnight and forms pyruvoyl 4-arm PEG. The protein of interest (eg. PD-1_{ecto}) C-terminal hydrazide chemoselectively reacts with pyruvoyl functionalized 4-arm PEG and results in site-specific C-terminal PEGylation of the protein via the resonance stabilized α-oxo hydrazone linkage.

FIG. 4 shows the binding specificity of the synthesized multivalent PEG PD-1_{ecto} pharmacophores to PD-L1 (FIG. 3A) and PD-L2 (FIG. 3B) coated plates. In contrast, only PD-L1 Fc chimera bound to CD80 coated plate (FIG. 3C), which was inhibited by the presence of 4-arm PEG-2k PD-1_{ecto} pharmacophore due to competitive binding to PD-L1 against CD80.

FIG. 5 presents evidence for selective binding of multivalent PEG PD-1_{ecto} pharmacophores to high-density PD-L1 plates. The pharmacophores of 4-arm PEG PD-1_{ecto} and 6-arm PEG PD-1_{ecto} exhibited strongly selective binding to high surface density of PD-L1. The discriminate binding between low and high density of PD-L1 was especially prominent for the pharmacophores with shorter linkers as shown with steeper curves. In contrast, the pharmacophores with longer linkers could also bind to the plate with lower density of PD-L1 and were less stringent in target density. The binding of anti-PD-L1 antibody to the plate was mainly in a linear mode (seen when X-axis in normal scale).

FIG. 6 demonstrates that multivalent pharmacophores possessed strong avidity to target overexpressing cells. In competitive binding to PD-L1 overexpressing SU-DHL-1 cells against PD-L1 blocking antibody, 4-arm PEG-2K PD-1_{ecto} and 6-arm PEG-3.4K PD-1_{ecto} showed IC₅₀ of 12.74 nM and 8.74 nM, respectively. Because the PD-L1 blocking antibody was used at ˜33.3 nM, 4-arm PEG-2K PD-1_{ecto} is about 2.6-fold more potent, and 6-arm PEG PD-1_{ecto} is about 3.8-fold more potent than the antibody, assuming there exists a linear relationship in the competitive binding assay.

FIG. 7 exhibits that multivalent pharmacophores, 4-arm PEG-SIRPα IgV and 6-arm PEG-SIRPα IgV, had much higher avidity than the affinity of wide-type SIRPα monomer. Although SIRPα Q67R mutant has ˜50% reduced affinity compared to the wide-type SIRPα, the multivalent pharmacophores with the mutant SIRPα IgV showed only slightly reduced avidity.

FIG. 8 shows the results of competitive binding assay on human red blood cells (RBCs). None of 4-arm PEG-SIRPα IgV and 6-arm PEG-SIRPα IgV, neither wide-type nor mutant SIRPα IgV, competed against CD47 blocking antibody in binding to RBCs until at the highest dose (200 nM). In contrast, anti-CD47 antibody bound to RBCs in a dose-dependent pattern.

FIG. 9 shows the results of hemagglutination assay. The multivalent PEG-SIRPα IgV pharmacophores showed no hemagglutination. In contrast, substantial hemagglutination was observed for CD47 blocking antibody, BRIC126.

FIG. 10 demonstrates that both multivalent PEG-SIRPα IgV pharmacophores substantially induced phagocytosis of Raji cells by macrophages. CD47 blocking antibody B6H12.2 was more potent than the multivalent pharmacophores in phagocytosis, likely due to the function of Fc domain of the antibody. However, the pharmacophores could enhance phagocytosis induced by rituximab when combined, suggesting that when a pro-phagocytic signal is present, blockade of CD47-SIRPα interaction by the pharmacophores can augment the phagocytic effect.

FIG. 11 demonstrates that the multivalent PEG-GFD pharmacophores had much higher binding avidity to uPAR than the affinity of uPA. In the competitive binding assay to uPAR overexpressing Hela cells between PE conjugated anti-uPAR antibody and 4-arm PEG-GFD, 6-arm PEG-GFD or uPA, 4-arm PEG-GFD and 6-arm PEG-GFD showed the IC₅₀ of 1.143 nM and 0.4117 nM, respectively, as compared to 34.89 nM for uPA.

FIG. 12 measures the activity of 4-arm PEG-GFD and 6-arm PEG-GFD to compete and dislodge uPA from uPAR on HT-1080 cells in which the overexpressed uPAR is occupied by uPA. Both multivalent PEG-GFD pharmacophores competed out the bound uPA from uPAR dose-dependently, with 6-arm PEG-GFD being more potent. The competitive process was also time-dependent and 3-hour was needed to reach close to maximal effect. In comparison, anti-uPAR antibody VIM5 was much less capable in the competition.

FIG. 13 demonstrates the ability of multivalent PEG-GFD pharmacophores to inhibit the adhesion of four uPAR overexpressing cell lines to vitronectin coated plate. In consistent with what has been observed for competitive binding assays (FIGS. 11 and 12), 6-arm PEG-GFD was more potent than 4-arm PEG-GFD. The uPAR blocking antibody VIM5 showed very limited inhibition of adhesion, even at the highest concentration.

FIG. 14 depicts the ability of 4-arm and 6-arm PEG-GFD pharmacophores to inhibit the chemotaxis of H1299 and TH-1080 cells. uPAR blocking antibody VIM5 was much less potent than the multivalent pharmacophores.

FIG. 15 shows the results of Matrigel invasion assay. Both 4-arm and 6-arm PEG-GFD pharmacophores significantly inhibited the Matrigel invasion of H1299 and HT-1080 cells at the dose of 10 nM. uPAR blocking antibody VIM5 also slowed the invasion, but was much less potent than the multivalent pharmacophores.

### DETAILED DESCRIPTION OF THE INVENTION

### 1. Introduction

A growing list of cell surface proteins and non-protein cell membrane components are reported overexpressed in a variety of cancers in cancer cells and/or non-cancer cells including immune cells in tumor microenvironment. The overexpression usually is functionally important for cancer immune evasion, carcinogenesis, cancer cell proliferation, cell migration, metastasis, resistance to apoptosis, and resistance to chemotherapeutic drugs and targeted cancer therapies. Therefore, the overexpression is closely correlated with the poor prognosis for many cancers. Overexpression of cell surface markers has also been found in other diseases such as infectious and inflammatory diseases. Below is a list of overexpressed cell surface proteins and glycolipids in cancers or non-cancer diseases. The list is not meant to be complete or exhaustive, but serves only as examples that they can be targeted by the multivalent pharmacophores as disclosed in this invention.

Overexpression of programmed cell death-ligand1 (PD-L1) has been found in tumor cells and/or immune cells in the tumor microenvironment in multiple cancer types including non-small cell lung cancer (NSCLC), renal cell carcinoma (RCC), melanoma, head and neck squamous cell carcinoma (HNSCC), gastric cancer, colorectal cancer (CRC), bladder cancer, and pancreatic cancer (Herbst et al., Nature, 2014, 515(7528):563-7; Powles et al., Nature, 2014, 515(7528):558-62). Programmed cell death-ligand2 (PD-L2) has also been reported overexpressing in many tumor types and present in stromal, tumor, and endothelial cells of the tumors (Yearlet et al., Clin Cancer Res, 2017, 23(12):3158-67; Jun et al., Cancer Res Treat, 2017, 49(1):246-54; Shin et al., Ann Surg Oncol, 2016, 23(2):694-702; Baptista et al., Hum Pathol, 2016, 47(1):78-84; Calles et al., J Thorac Oncol, 2015, 10(12):1726-35). As reviewed by Sun, et al. (Immunity, 2018, 48(3):434-52), co-amplification of PD-L1 and PD-L2 in different types of tumors are observed and exposure to type I interferons has a much greater effect on expression of PD-L2 than PD-L1 in melanoma cells. High expression of programmed cell death-1 (PD-1) in T cells and other tumor-infiltrating lymphocytes is well known and indicates an exhausted phenotype for effector T cells (Ohaegbulam et al., Trends Mol Med, 2015, 21(1):24-33).

Just like what has been observed in cancers, the phenomenon of T-cell exhaustion exists in chronic infectious diseases. Up-regulation of PD-1 and other immune checkpoint molecules are observed in T-cells and other immune cells in patients with chronic viral, bacterial, or parasitic infections including tuberculosis, malaria, and patients infected with human immunodeficiency virus (HIV), hepatitis B virus (HBV) and hepatitis C virus (HCV) (Rao et al., Int J Infect Dis, 2017, 56:221-8; Attanasio et al., Immunity, 2016, 45(5):1052-68). There are evidences suggesting that PD-1/PD-L1 blockage can offer beneficial effects to these diseases.

Besides PD-L1, other members of B7 family can be aberrantly expressed in different tumor entities. B7-H3 and B7-H4 are overexpressed in prostate cancer, pancreatic cancer, breast and ovarian cancers, RCC, melanoma, esophageal squamous cell carcinoma, and lung cancers among others. In prostate cancer, both B7-H3 and B7x are highly expressed with 93% and 99%, respectively, of tumors having aberrant expression (Roth et al., Cancer Res, 2007, 67(16):7893-900; Zang et al., PNAS, 2007, 104(49):19458-63). Patients with strong intensity for B7-H3 and B7x are significantly more likely to have disease spread at time of surgery and poor prognosis. Positive expression of B7-H4 is observed in more than 90% esophageal squamous cell carcinoma, and the level of expression correlates with the degree of disease spread, with higher expression predicting lower density of infiltrating T cells and worse outcome (Chen et al., Cancer Immunol Immunother, 2011, 60(7):1047-55). B7-H3 and B7-H4 are overexpressed in primary and metastatic melanoma, and a survival benefit for patients with B7-H4 low expressing melanoma is found (Quandt et al., Clin Cancer Res, 2011, 17(10):3100-11).

Galectins, a family of glycan-binding proteins, have emerged as novel regulatory checkpoints that promote immune evasive programs by inducing T-cell exhaustion, limiting T-cell survival, favoring expansion of regulatory T cells, de-activating natural killer cells and polarizing myeloid cells toward an immunosuppressive phenotype (Thijssen et al., Biochi Biophys, 2015, 1855(2):235-47; Rabinovich et al., J Mol Biol, 2016, 428(16):3266-81; Mendez-Huergo et al., Curr Opin Immunol, 2017, 45:8-15). Galectin-1 tilts the balance of the immune response toward a Th2 profile by selectively deleting Th1, Th17, and CD8+ T cells. Moreover, it drives the differentiation of T regulatory cells (T_{regs}), endows dendritic cells (DCs) with tolerogenic potential, polarizes macrophages toward an anti-inflammatory M2-type profile, inhibits NK cell recruitment, and limits transendothelial T-cell migration. Galectin-3 acts by restricting T cell receptor (TCR)-mediated signaling and promoting T-cell anergy and exhaustion by distancing the TCR from CD8 and engaging LAG-3 on the surface of CD8+ T cells. In addition, this lectin impairs the antitumor activity of NK cells by inhibiting NKp30-mediated cytotoxicity and interrupting NKG2D-MICA interaction. Galectin-3 may also control the expansion of tumor-associated plasmacytoid DCs. Galectin-9 confers immune privilege to tumor cells through Tim-3 dependent or independent mechanisms. It binds to CD44 and cooperates with TGF-β1 to promote T_{reg} cell differentiation and favors expansion of immunosuppressive MDSCs. Malignant transformation is frequently associated with increased expression of galectins, most notably galectin-1 and galectin-3, in cancer cells and immune and/or stromal cells. Increased galactin-1 expression is a common phenomenon in most cancer types and is associated with poor overall survival and disease free survival, irrespective of the cancer type. The overexpression of galactin-3 is observed in many cancers of digestive tract and urinary systems, and in thyroid cancer and melanoma. Galectins are known to be secreted. Increased levels of circulating galectin-1 and -3 are often in agreement with altered tissue expression in cancers, and could serve as diagnostic and/or prognostic markers for some cancers.

T cell immunoglobulin and mucin-domain containing molecule 3 (Tim-3) is a co-inhibitory receptor that is expressed on cytotoxic lymphocytes, FoxP3+T_{reg} cells, NK cells, monocytes, macrophages, and dendritic cells (Das et al., Immunol Rev, 2017, 276(1):97-111). Tim-3 plays a key role in inhibiting Th1 responses and the expression of cytokines such as TNFα and INFγ. High level of Tim-3 expression is correlated with T cell exhaustion, and Tim-3+PD-1+CD8+ T cells represent a “deeply” exhausted T cell population as compared to PD-1+ single positive CD8+ T cells. In addition to effector T cells, Time-3 has been shown to enhance the regulatory function of FoxP3+T_{regs}. The immune inhibitory function of Tim-3 extends to other immune cells, including NK/NKT cells, dendritic cells, and macrophages. Tim-3 serves as a checkpoint receptor in tumor immunity. It has been shown that in vivo blockade of Tim-3 with checkpoint inhibitors enhances anti-tumor immunity and suppresses tumor growth in several preclinical tumor models. The increased expression of Tim-3 has been found in multiple tumors, including expression on cancer cells and tumor infiltrating T cells, T_{regs} and tumor-associated macrophages, and indicates poor prognosis and more advanced tumor grades (Liu et al., J Hematol Oncol, 2018, 11(1):126; Burugu et al., Oncoimmunology, 2018, 7(11):e1502128).

The role of Tim-3 in infectious diseases is now appreciated on exhausted T cells from a variety of chronic infections including HIV, HBV and HCV (Attanasio et al., Immunity, 2016, 45(5):1052-68). Tim-3 is one of immune regulatory factors involved in HBV infection (Liu et al., World J Gastroenterol, 2016, 22(7):2294-2303). In chronic HBV infection, its expression is elevated in many types of immune cells, such as T helper cells, cytotoxic T cells, dendritic cells, macrophages and natural killer cells. Tim-3 overexpression is often accompanied by impaired function of these immune cells.

CD74, an evolutionarily conserved type II membrane protein, participates in several key process of the immune system, including antigen presentation, B-cell differentiation and inflammatory signaling. The overexpression of CD74 is observed in hematological malignancies and various solid tumors and has been suggested to serve as a prognostic factor, with higher relative expression of CD74 behaving as a marker of disease progression (Shachar et al., Leuk Lymphoma, 2011, 52(8):1446-54; Borghese et al., Expert Opin Ther Targets, 2011, 15(3):237-51; Greenwood et al., J Proteomics, 2012, 75(10):3031-40). Experimental data show that macrophage migration inhibitory factor (MIF), an inflammatory cytokine, is overexpressed in breast cancer cells and interacts with its main receptor CD74 and its co-receptor CXCR4, both overexpressed in breast cancer cells, promoting survival, neo-angiogenesis and inhibiting autophagy (Richard et al., Int J Oncol, 2015, 47(5):1627-33).

CD47, also known as integrin-associated protein, is a ubiquitously expressed glycoprotein of the immunoglobulin superfamily and plays a critical role in self-recognition (Veillette and Chen, Trends Immunol, 2018, 39(3)173-84; Weiskopf, Eur J Cancer, 2017, 76:100-109). CD47 interacts with signal regulatory protein-alpha (SIRPα), highly expressed in myeloid cells such as macrophage and dendritic cells, and deliver an anti-phagocytic ‘don't eat me’ signal. Therefore, CD47-SIRPα axis constitutes an innate immune checkpoint. Various solid and hematological cancers overexpress CD47, which forms part of the mechanism of tumor immunological evasion. Increased expression of CD47 is also associated with poor prognosis in these patients. Studies have shown that blockade of CD47-SIRPα interaction enhances the phagocytic activity of phagocytes and promotes the stimulation of tumor-specific cytotoxic T cells. Because CD47 is expressed on most cell types, on-target/off-tumor side effect of anti-CD47 antibody on normal cells, in particular erythrocytes and platelets, is a serious concern with regard to such treatment. Similar to the increased expression of CD47 in many types of cancer, SIRPα is also prominently expressed in tumor tissues from patients with renal cell carcinoma or melanoma compared with the surrounding normal tissues.

CD24, also known as heat stable antigen or small-cell lung carcinoma cluster 4 antigen, is a glycosylphosphatidylinositol-anchored surface protein. CD24 is highly expressed in multiple cancers, including ovarian cancer and triple-negative breast cancer (Barkal et al., Nature, 2019, 572(7769):392-6). Stratification of cancer patients by CD24 expression revealed increased relapse-free survival or overall survival advantage with lower CD24 expression. CD24 expressed in tumor cells interacts with inhibitory receptor sialic-acid-binding mg-like lectin 10 (Siglec-10) which is expressed in a substantial fraction of tumor-associated macrophages (TAMs) and inhibits phagocytosis of the tumor cells. Therefore, CD24-Siglec-10 axis forms another innate immune checkpoint. Interestingly, the expression of CD24 and CD47 seems to be inversely related among patients with diffuse large B cell lymphoma. Genetic ablation of either CD24 or Siglec-10, as well as blockade of the CD24-Siglec-10 interaction using antibody, robustly augments the phagocytosis of all CD24-expressing tumor cells. Animal models show that CD24 deletion or blockade with antibody results in a macrophage-dependent reduction of tumor growth and increased survival.

The chemokine/receptor axis of SDF-1 and CXCR4 normally plays a critical role in the homing and retention of hematopoietic stem cells and lymphocytes in the bone marrow and trafficking of these cells to the sites of tissue inflammation and damage. CXCR4 overexpression has been reported in many types of cancers. Preclinical and clinical studies suggest that the CXCR4/SDF-1 axis plays an important role in the metastasis of many cancers, including breast, ovarian, colorectal, head and neck, lung and pancreatic carcinoma. In addition, it has been found that overexpression of CXCR4 in tumor tissue predicts a worse outcome in patients who have breast cancer (Xu et al., Drug Des Devel Ther, 2015, 9:4953-64), esophageal cancer (Wu et al., Tumour Biol, 2014, 35(4):3709-15) and stage IV non-small cell lung cancer (Otsuka et al., J Thorac Oncol, 2011, 6(7):1169-78).

The folate cycle sustains key metabolic reactions and is essential for rapidly growing cells. Folate receptors (FRs) are high-affinity folate transporters (Ledermann et al., Ann Oncol, 2015, 26(10):2034-43). Folate receptor-α (FRα) is overexpressed in a majority of tumors of the ovary, uterus, ependymal and brain, and malignant pleural mesotheliomas. It is also overexpressed in a variable percentage of lung, kidney, breast, and colon carcinomas. In vitro and in vivo studies have demonstrated a correlation between human ovarian cancer growth and FR overexpression. Inhibition of FRα expression in FRα-positive tumor cell lines suppresses cell proliferation. Furthermore, FRα expression may also induce drug resistance by enhancing the anti-apoptotic ability of tumor cells.

Transferrin receptor (TfR, CD71) is a type II transmembrane homodimer glycoprotein involved in the cellular uptake of iron via internalization of iron-loaded transferrin (Tf). Each monomer of TfR contains a large extracellular C-terminal domain that contains Tf-binding site, a single pass transmembrane domain, and a short intracellular domain. Iron is a nutrient essential for cell growth and iron-requiring metabolic processes including DNA synthesis, energy metabolism, detoxification and antioxidant defense (El Hout et al., Semin Cancer Biol, 2018, 53:125-38). Consequently, rapidly growing cells require more iron for their growth than resting cells. Not surprisingly, TfR is expressed at greater levels in various cancer cells including breast, lung, colorectal, bladder and pancreatic cancers, and in many hematological cancers, and the expression is correlated with tumor grade and stage or prognosis (Daniels et al., Clin Immunol, 2006, 121(2):144-58). In ER+ breast cancer patients, overexpression of TfR is associated with resistance to hormonal therapy, higher grade and poor clinical outcome (Habashy et al., Breast Cancer Res Treat, 2010, 119(2):283-93). Interestingly, extensively cross-linking of TfR, either by IgM antibody or by secondary anti-IgG antibody to the IgG anti-TfR treated cells, inhibits the cell growth due to blockage of internalization of TfR and iron uptake (Daniels et al., Clin Immunol, 2006, 121(2):144-58).

Epidermal growth factor receptor (EGFR or ErbB-1/HER1) belongs to the ErbB family of receptor tyrosine kinases which also includes ErbB-2 (HER2), ErbB-3 (HER3), and ErbB-4 (HER4) (Normanna et al., Gene, 2006, 366(1):2-16). Binding of ligands to the extracellular domain of ErbB receptors induces the formation of receptor homo- or hetero-dimers, and subsequent activation of the intrinsic tyrosine kinase domain, leading to activation of intracellular signaling pathways. Overexpression of EGFR induces transformation in the presence of appropriate levels of ligands, and maintains autonomous proliferation of cancer cells. In addition, tumors that co-express different ErbB receptors are often associated with a more aggressive phenotype and a worse prognosis. Expression of EGFR and ErbB-3 occurs in the majority of human carcinomas at high frequency. On average, 50% to 70% of lung, colon and breast cancers have been found to overexpress EGFR or ErbB-3. HER2 overexpression is generally more restricted, with approximately 30% of human primary breast cancer expressing this receptor. However, other malignancies of epithelial origin also show significant rates of HER2 positivity such as bladder, esophageal, and gallbladder cancers (Yan et al., Cancer Metastasis Rev, 2015, 34(1):157-64). Multiple carcinomas have been reported with HER3 overexpression, including melanoma, cervical, ovarian, colorectal, gastric and breast cancers (Ocana et al., J Natl Cancer Inst, 2012, 105(4):266-73). Co-overexpression of HER2 and HER3 likely predicts much worse prognosis. The expression of ErbB-4 has been investigated in breast and colon cancers, where this receptor is expressed in approximately 50% and 22% of the tumors, respectively. Glioblastoma multiforme (GBM) often exhibits overexpression of EGFR, with a frequency of about 40% in primary GBM (Gan et al., J Clin Neurosci, 2019, 16(6):748-54). Of the GBM that overexpresses EGFR, 63% to 75% are also found to have rearrangements of the EGFR gene, resulting in tumors expressing both wild-type and mutated EGFR. The most common of these EGFR mutants is the EGFRvIII. The truncated extracellular domain of EGFRvIII is unable to bind any known EGFR ligand. However, the mutant receptor shows constitutive kinase activity, with impaired endocytosis and degradation due to inefficient ubiquitination and rapid recycling. In addition, EGFRvIII signaling may trigger different outcomes to that of the wide-type receptor, as it can signal through EGFRvIII homodimers or through heterodimers with either EGFR or HER2.

Platelet-derived growth factor (PDGF) signaling system is activated by binding of four PDGF polypeptide chains denoted PDGF-A, —B, —C and -D that make up five functional growth factors, PDGF-AA, -BB, -AB, —CC and -DD, to their corresponding receptors PDGFRα and PDGFRβ (Papadopoulos et al., Mol Aspects Med, 2018, 62:75-88; Heldin et al., J Intern Med, 2017, 283(1):16-44). PDGF family has important roles during embryogenesis, particularly in the development of various mesenchymal cell types in different organs. In the adult, PDGF stimulates wound healing and regulates tissue homeostasis. PDGFRs and their ligands have been found to be overexpressed or misregulated in many cancers, correlating with reduced overall survival. For example, amplification of PDGFRA gene has been observed in 5-10% glioblastoma multiforme, resulting in the expression of high amount of the receptor at the cell surface. PDGFRα and β are overexpressed in advanced hepatocellular carcinoma and correlate with poor prognosis. PDGFRβ is overexpressed in a majority of primary and metastatic prostate cancer in the tumor stromal cells, and correlates to clinical relapse. In some types of cancers, such as gliomas, sarcomas, lymphocyte leukemias, and dermafibrosarcoma protuberans (DFSP), there is a co-expression of ligands and receptors in the transformed malignant cells. In other cancer types, PDGFRs are expressed on non-cancerous cells of the tumor microenvironment or tumor stroma that are able to crosstalk with the cancer cells and thus constitute an important factor in the development and pathophysiology of the tumors.

Signaling through its receptors FGFR1, FGFR2, FGFR3 and FGFR4, fibroblast growth factor (FGF) regulates cell fate, angiogenesis, epithelial-to-mesenchymal transition (EMT), immunity, and metabolism (Katoh, Trends Pharmacol Sci, 2016, 73(12):1081-96; Haugsten et al., Mol Cancer Res, 2010, 8(11):1439-52). FGFRs are overexpressed as cancer drivers due to FGFR gene amplifications, altered distal FGFR enhancers, and other genetic alterations in FGFR trans-regulation. FGFR1 is overexpressed in estrogen-positive breast, gastric, lung, ovarian, and urothelial cancers. FGFR2 is upregulated in triple-negative breast cancer and gastric cancer. FGFR3 is overexpressed in ovarian and urothelial cancers, and in multiple myeloma and T-cell lymphoma. FGFR4 is upregulated by PAX3-FOXO1 cancer driver of rhabdomyosarcoma. FGFR1 is often co-overexpressed with other co-amplified genes such as NSD3 in breast cancer and lung cancer. By contrast, NSD2 and FGFR3 at human chromosome 4p16.3 are overexpressed as cancer drivers in multiple myeloma with t(4; 14)(p16; q32).

Anaplastic lymphoma kinase (ALK) is a receptor tyrosine kinase that has been implicated in the pathogenesis of a variety of cancers (Cao et al., Cancers (Basel), 2017, 9(9), pii:E123; Hallberg et al., Nat Rev Cancer, 2013, 13(10):685-700). Amplification and overexpression of ALK protein has been reported in different types of cancers, including melanoma, NSCLC, neuroblastoma, rhabdomyosarcoma, ovarian cancer, and breast cancer. ALK overexpression has been shown to activate the Ras-ERK pathway to promote cell proliferation, and the JAK3-STAT3 and PI3K-AKT pathways to increase cell survival.

The ephrin (Eph) receptors are the largest family of receptor tyrosine kinases and they mainly regulate cell proliferation and migration during development as well as tissue homeostasis (Zhou et al., Biol Pharm Bull, 2017, 40(10):1616-24; Brantley-Sieders et al., PLoS One, 2011, 6(9):e24426). Eph family of receptors is divided into class A and class B based on sequence homology and binding affinity for two distinct types of membrane-anchored ephrin ligands. There are 14 receptors (9 class A and 5 class B) and 8 ligands (5 class A and 3 class B). Because of overlapping expression pattern, promiscuous interaction between ligands and receptors, bidirectional signaling between ligands and receptors, and pleiotropic functions, the role of Eph receptor/ephrin system is extremely complex. Eph receptor A2 (EphA2) is the most widely characterized member of Eph receptors. Overexpression of EphA2 is reported in various solid tumors, such as melanoma, breast, ovary, prostate, pancreas, glioblastoma, head and neck, renal, lung, bladder, gastric, esophageal, colorectal, and cervical cancers. For example, EphA2 is expressed at low levels in normal breast epithelium, and overexpressed in 60-80% of breast cancers. In many cases, the expression of EphA2 is associated with a more aggressive cancer phenotype and correlated with tumor metastasis and poor patient survival. However, some studies found that EphA2 reduces cancer cell proliferation and motility, suggesting that EphA2 has both pro- and anti-oncogenic functions.

Insulin-like growth factor (IGF) signaling pathway is a complex network that plays an important role in regulating growth and development in normal human tissues (Simpson et al., Target Oncol, 2017, 12(5):571-97; lams et al., Clin Cancer Res, 2015, 21(19):4270-7). Insulin/IGF axis comprises three ligands [insulin, IGF ligand-1 (IGF-1), and IGF ligand-2 (IGF-2)], and multiple receptors [insulin receptor-A and -B (INSR-A, INSR-B), IGF-1 receptor (IGF-1R) and IGF-2 receptor (IGF-2R), and hybrid receptors, INSR-A/B, IGF-1R/INSR-A, and IGF-1R/INSR-B], IGF binding proteins (IGFBPs), IGFBP-specific proteases, and IGFBP-related peptides. Activation of IGF-1R, INSR-A, and IGF-1R/INSR hybrid receptors by IGF-1, IGF-2, and insulin promotes cellular growth, survival, and metastasis of cancers. Overexpression of IGF-1R and INSR-A has been observed in various cancers, including Ewing sarcoma, breast, prostate, colorectal, lung and pancreatic cancers, and melanoma. This overexpression is associated with faster disease progression and a poor prognosis in some tumors. Moreover, the presence of a functional IGF-1R has been shown to be essential for malignant transformation. In addition, IGF signaling in tumor cells is also driven by overexpression of IGF-1 and IGF-2. As a result, any attempt that target one receptor of IGF axis will not likely to succeed in cancer therapy. Furthermore, preclinical data in breast cancer suggest that inhibition of IGF-1R upregulates INSR-A, leading to IGF-2-induced amplification of Wnt and Notch signaling.

Chemotherapeutics are still effective and widely used treatment options for metastatic tumors. However, most cancer cells will develop resistance simultaneously to several structurally unrelated drugs that do not have a common mechanism of action, a phenomenon known as multidrug resistance (MDR). MDR significantly limits the effectiveness of chemotherapy in cancers. MDR is caused by overexpression of a large family of ATP-dependent efflux pumps, i.e. the ATP-binding cassette (ABC) transporters. This superfamily is composed of 48 genes and divided into 7 distinct subfamilies (Gottesman et al., Nat Rev Cancer, 2002, 2(1):48-58; Genovese et al., Drug Resist Updat, 2017, 32:23-46; Mohammad et al., Biomed Pharmacother, 2018, 100:335-48). ABC transporter proteins are important cell surface proteins and responsible for transportation of different endogenous ligands such as lipids, peptides, proteins, sterols, drugs, and a large variety of primary and secondary metabolites. Three ABC transporters have been studied the most, P-glycoprotein (P-gp/MDR1/ABCB1), breast cancer resistance protein (BCRP/MXR/ABCG2), and multidrug resistance protein 1 (MRP1/ABCC1). P-gp overexpression has been found in many solid tumors such as colon, kidney, ovary, breast, adrenocortical and hepatocellular cancers. P-gp expression has also been reported in acute myelogenous leukemia from about one-third of patients at the time of diagnosis, and more than 50% of patients at relapse. P-gp expression can be increased up to 1000-fold in lung cancer cells with acquired paclitaxel resistance. BCRP expression has been associated most consistently with poorer outcomes in acute myelogenous leukemia, acute lymphoblastic leukemia, and breast and lung cancer. MRP1 is highly expressed in leukemias, esophageal carcinoma and non-small cell lung cancer. Similarly, overexpression of MRP1 is strongly predictive of poor outcome in neuroblastoma patients.

Claudins are a family of tight junction proteins regulating paracellular permeability and cell polarity with different patterns of expression in benign and malignant human tissues (English et al., Int J Mol Sci, 2013, 14(5):10412-37). Normal epithelial cells are held together by tight junctions (TJs), adherens junctions (AJs), and gap junctions. There is evidence that disruption of the cell to cell adhesion is a critical step in the process of cellular transformation and tumor cell metastasis. The role of the claudins in this process is continuously being explored with new discoveries still occurring. Apart from contributing to mechanical cell adhesion at epithelial and endothelial cell interfaces, claudins also have the capacity to recruit cell signaling proteins and as such may regulate cell proliferation, differentiation and subsequent neoplastic transformation. There are 27 different types of claudins identified with varying cell- and tissue-specific expression. Deregulation of the mitogen-activated protein kinase (MAPK) pathway can lead to the mis-localization of claudins and other TJ proteins. The delocalization of claudin proteins from cell membranes is common among transformed cells and in ovarian cancer which is associated with tumor cell migration and invasion. The pattern of expression of claudins in normal tissues, benign and malignant tumors is not only complex but also organ dependent. Claudin-1, -3, -4, -5, and -7 are the most commonly overexpressed in ovarian cancer. Overexpression of claudin-1 in colon cancer, claudin-10 in hepatocellular carcinoma, and claudin-18 in gastric cancer has been reported.

The epithelial cell adhesion molecule (EpCAM) is a pleiotropic type I transmembrane glycoprotein that was first described as an epithelial-specific intercellular adhesion molecule. However, many data suggest that its role is not limited to cell adhesion, and it's expressed not only in epithelial cells, but also in various tissue stem cells, precursors, and in embryonic stem cells (Heerros-Pomares et al., Crit Rev Oncol Hematol, 2018, 126:52-63). EpCAM is frequently overexpressed in epithelial tumors, in contrast to its low expression in normal simple epithelia. The functional duality of EpCAM has been reported for cancers. On one hand, EpCAM mediates hemophilic adhesive interactions and acts as a tumor suppressor. In line with this, loss of EpCAM has been associated with increased migratory potential, and its expression in metastases seems lower compared to primary tumors. On the other, several oncogenic functions of EpCAM has been discovered, including abrogation of E-cadherin-mediated cell-cell adhesion and association with claudin-7, which interferes with homotypic cell-cell adhesion, and the promotion of cell motility, proliferation, survival, carcinogenesis and metastasis through the Wnt pathway. The extracellular domain of EpCAM can be cleaved and is detectable in serum from cancer patients including breast, ovarian, lung, colorectal, and prostate cancers.

Carcinoembryonic antigen-related cell adhesion molecules (CEACAM) are a family of highly glycosylated transmembrane proteins of the immunoglobulin superfamily (Lee et al., Gastroenterol Res Pract, 2017, 2017:7521987; Rizeq et al., Cancer Sci, 2018, 109(1):33-42). CEACAM have 12 members, and CEACAM5 (CEA) and CEACAM6 are among the best characterized in cancer process. CEA and CEACAM6 are linked to cell membrane by glycosylphosphatidylinositol anchor. In healthy adults, the expression of CEA is mostly found in columnar epithelial cells and mucus-secreting cells of the colon. CEA can also be found at a lower level in secretory epithelial cells present in the stomach and small intestine, in epithelial cells of the prostate, in secretory epithelial and ductal cells of the sweat glands, in transitional epithelial cells of the urinary bladder, as well as in squamous epithelial cells of the esophagus, tongue and cervix. Overexpression of CEA is associated with many types of cancers including gastrointestinal, respiratory, and genitourinary system and breast cancer. The membrane-anchoring of CEA can be cleaved and the soluble CEA circulates through blood vessels, and is used to monitor recurrence of pancreatic and colorectal cancers and prognosis. The expression pattern of CEACAM6 in normal tissues is similar to CEA with a few exceptions. CEACAM6 is not expressed in small intestine, but in duct cells of the breast and pancreas, in myeloid cells of the bone marrow and spleen, and in pneumocytes and bronchiole epithelial cells of the lung. Overexpression of CEACAM6 is observed in more than 50% of human adenocarcinomas, including colorectal cancer, pancreatic ductal adenocarcinoma, and breast cancer. Overexpression of CEA and CEACAM6 promotes cancer cell's invasion, metastasis, and resistance to therapeutic agents and apoptosis.

CD44 is a non-kinase transmembrane glycoprotein expressed in various cell types. CD44 activates and modulates a number of cell signaling networks, and plays important roles in tumor progression, metastasis and chemoresistance (Chen et al., J Hematol Oncol, 2018, 11(1):64; Karousou et al., Matrix Biol, 2017, 59:3-22). In humans, CD44 is encoded by 19 exons with 10 of these exons constant in all isoforms. The standard form of CD44 (CD44s) is encoded by the ten constant exons. CD44 variant isoforms (CD44v) are generated by alternative splicing and possessing the ten constant exons and any combination of the remaining nine variant exons. The encoded CD44 peptide can be further modified by N- and O-linked glycosylation and glycosaminoglycanation by addition of heprin sulfate or chondroitin sulfate. CD44 can undergo isoform switching in tumor cells. Overexpression of CD44, particularly CD44v isoforms, has been found in different types of cancers, including gastric, colorectal, pancreatic, head and neck, and non-small cell lung cancers. CD44 expression in colorectal cancer is correlated with poor overall survival, poor differentiation, and lymph node and distant metastasis (Wang et al., Front Oncol, 2019, 9:309). Elevated expression of CD44 in pancreatic cancer is reported to play a role in metastasis, aggressive malignant behaviors and patient survival (Li et al., Int J Clin Exp Pathol, 2015, 8(6):6724-31). CD44 expression in head and neck cancer is related to worse TNM stage, tumor grade and prognosis in pharyngeal and laryngeal cancers (Chen et al., BMC Cancer, 2014, 14:15). The expression of CD44 is upregulated in the leading subpopulation of invading breast cancer cells and efficiently promotes the collective invasion into adjacent tissue (Yang et al., Oncogen, 2019, 38(46):7113-32). Accumulating evidence indicates that CD44, especially CD44v isoforms, are cancer stem cell (CSC) markers and critical players in regulating the properties of CSC (Yan et al., Stem Cells Transl Med, 2015, 4(9):1033-43). CD44 binds to several ligands including hyaluronic acid (HA), osteopontin (OPN), chondroitin, collagen, fibronectin, fibrin, laminin, matrix metalloproteinases (MMPs), and serglycin/sulfated proteoglycan. HA is a major component of extracellular matrix and is the most specific ligand for CD44. HA binding to CD44 causes conformational changes favoring the binding of adaptor molecules to the intracellular cytoplasmic tail of CD44, leading to cell signaling that enhances cell adhesion, migration, and proliferation. The pathways activated through CD44-HA binding include Ras, MAPK and PI3K. In breast cancer cells, HA activation of CD44 leads to the expression of multidrug resistance gene P-gp and anti-apoptosis gene BcL2. OPN binds to CD44 and promotes tumor progression and metastasis. Versican (VCAN) is a chondroitin sulfate proteoglycan that binds to HA leading to structural aggregations of these molecules. Elevated level of VCAN correlates with higher tumor grade and invasiveness in breast cancer, and promotes the motility and invasion of ovarian cancer cells.

As the main cell adhesion receptors for components of the extracellular matrix (ECM), integrins are a family of 24 transmembrane heterodimers generated from a combination of 18α integrin and 8β integrin subunits. Various studies identified integrins, in particular those of the αV family, are relevant to cancer cell proliferation, survival, migration, invasion and metastasis, and promote tumor angiogenesis, matrix remodeling, and recruitment of immune and inflammatory cells (Alday-Parejo et al., Cancers, 2019, 11(7), pii:E978; Hamidi and Ivaska, Nat Rev Cancer, 2018, 18(9):533-48). In addition, integrin may be involved in regulating PD-L1 expression and anticancer immune response (Vannini et al., Proc Natl Aca Sci, 2019, 116(40): 20141-50). Integrins bind to insoluble ECM proteins (e.g., fibronectins, laminins, collagens), matricellular proteins (e.g., Cyr61/CTGF/NOV, CCN), cell surface proteins (e.g., ICAM, VCAM-1), and soluble ligands (e.g., fibrinogen, complement proteins, VEGF, FGF2, angiopoietin-1, TGFβ). Altered integrin expression has been linked to many types of cancer and is associated with the extent of neoplastic progression, patient survival or response to therapy. Many integrins are reported overexpressed in cancer cells and stromal cells in the tumor microenvironment, including ovarian, breast, prostate, colorectal, liver and gastric cancers as well as non-small cell lung cancer, glioblastoma, and multiple myeloma.

One of the major events that underlie metastasis is the proteolytic degradation of the extracellular matrix (ECM) to promote tumor cell invasion, migration, and homing to distant organs. Even though several protease systems are implicated in this process, a large body of evidence has identified the urokinase-type plasminogen activator receptor (uPAR) system as a central player in mediating proteolysis during cancer invasion and metastasis (Mahmood et al., Front Oncol, 2018, 8:24). uPAR is a single polypeptide chain with its C-terminal end covalently connected to the cell membrane by a glycosylphosphatidylinositol anchor. Binding of uPA and its zymogen pro-uPA to uPAR increases their ability to convert plasminogen to plasmin. In addition, uPAR cross-talks with integrins, vitronectin, G-protein coupled receptors, and receptor tyrosine kinases to regulate cancer cell dormancy, proliferation and angiogenesis, and contribute to epithelial-mesenchymal transition (EMT). The elevated expression of uPAR in cancer cells is regulated by different mechanisms, including co-amplification with HER2 in breast cancer. Higher expression of uPA is also reported and correlated with poor outcome in prostate, endometrial, colorectal, hepatocellular, pancreatic, gastric, and head and neck cancers, and acute myeloid leukemia.

Cell surface proteases have long been implicated in carcinogenesis. Among them, type II transmembrane serine proteases (TTSPs) are overexpressed in multiple tumors and are critical for the remodeling of tumor extracellular matrix and activation of oncogenic signaling pathways (Tanabe et al., FEBS J, 2017, 284(10):1421-36). Matriptase is among the most studies members of the TTSP family and is normally expressed in the epithelial compartment in a wide variety of tissues. Matriptase is shown to be overexpressed in many types of epithelial tumors including the breast, ovary, uterus, prostate, colon, cervix, and skin cancers. An important finding in several cancers is that the ratio of matriptase to its endogenous inhibitors, hepatocyte growth factor activator inhibitor (HAI)-1 and HAI-2, is increased, suggesting that the balance of protease activity can be shifted, leading to unopposed active matriptase, ultimately causing detrimental procarcinogenic effects. Hepsin, another member of TTSPs, is shown to be consistently expressed and upregulated in prostate cancer, and high levels in the tumor are indicative of poor outcome and relapse after radical prostatectomy (Stephan et al., J Urol, 2004, 171(1):187-91). Hepsin expression has also been well-documented in several other types of cancers, including ovarian and breast cancers. For example, hepsin is overexpressed in 40-50% of luminal A, B, and HER2+ types of breast cancer, and up to 60% of triple negative breast cancer. Furthermore, hepsin is predominantly expressed as the processed active form. Another member of TTSPs, TMPRSS2, may promote metastasis, like hepsin. TMPRSS2 gene expression is several folds higher in prostate cancer cells compared to benign prostate tissue.

Increasing evidence implicates serine proteinase, such as hepsin, in the proteolytic cascades leading to the pathological destruction of extracellular matrices such as cartilage in osteoarthritis (OA). The expression of hepsin is upregulated in OA and correlates with severity of synovitis (Wilkinson et al., Sci Rep, 2017, 7(1):16693). Proteoglycans (PGs) are major components of extracellular matrix (ECM) and play key roles in ECM structural organization and cell signaling, contributing to the control of numerous normal and pathological processes (Theocharis and Karamanos, Matrix Biol, 2019, 75-76:220-59). Cell surface associated PGs include chondroitin sulfate proteoglycan 4 (CSPG4), glypicans and syndecans, whose expression is markedly affected in cancer and stromal cells in tumors. They are actively involved in cell-cell and cell-matrix interactions and signaling affecting cancer cell proliferation, spreading and angiogenesis. CSPG4 is a single pass type I transmembrane protein. Up-regulation of CSPG4 is a frequent event in tumor progression and it is accumulated in several tumors such as melanoma, glioblastoma, breast and pancreatic cancers, head and neck squamous cell carcinoma, and acute myeloid and lymphoblastic leukemia. Glypicans bind to plasma membrane via a glycosylphosphatidylinositol anchor covalently bound to their C-terminus. There are six members of glypicans (glypican-1 to -6). Glypican-1 is up-regulated in cancers including breast, pancreas, esophagus and brain, and is associated with poor prognosis and chemoresistance in patients (Lu et al., Cancer Med, 2017, 6(6):1181-91). Glypican-2 is overexpressed in neuroblastoma. Glypican-3 exhibits diverse biological roles—some cancers show no expression of glypican-3 and others demonstrate up-regulation such as in hepatocellular carcinoma (Montalbano et al., Oncol Rep, 2017, 37(3):1291-1300). It seems the same has been reported for glypican-5. For example, glypican-5 is overexpressed in salivary adenoid cystic carcinoma and rhabdosarcoma, and down-regulated in hepatocellular, prostate and lung cancer. The family of syndecans consists of four types of transmembrane PGs (syndecan-1 to -4). Syndecans are ubiquitously expressed in all cells, except for erythrocytes. High levels of syndecan-1 on tumor cells are correlated with disease progression in pancreatic, ovarian and thyroid cancer, and in multiple myeloma, Hodgkin lymphoma and liposarcoma. In contrast, loss of cell surface syndecan-1 is observed in some other cancers. Syndecan-2 is up-regulated in several malignancies including lung, ovarian, colon, prostate, esophageal squamous cell carcinoma, melanoma, brain tumors, osteosarcoma and mesothelioma. Syndecan-4 has been found to be overexpressed in breast cancer, osteosarcoma, colon cancer, melanoma, malignant T-cells in Sezary syndrome, and testicular germ cell tumors.

Mucins are a family of glycosylated proteins with high molecular weight and complex molecular organization expressed on the epithelia (Jonckheere et al., Biochimi, 2010, 92(1):1-11; Chugh et al., Biochim Biophys Acta, 2015, 1856(2): 211-25). The glycan moieties on mucins serve as ligands for various carbohydrate-binding proteins, such as galectins, selectins, and siglecs, and mediate diverse biological processes including cell adhesion, migration, trafficking, and inflammation. Aberrant expression of mucins is observed in various malignancies wherein they play an essential role in cancer pathogenesis. For example, MUC1 is involved in breast cancer pathogenesis as it affects several signaling pathways that influence disease aggressiveness. The C-terminal subunit of MUC1, also known as MUC1-C, acts as an oncoprotein through its interaction with various receptor tyrosine kinases such as EGFR and ErbB2, which leads to the activation of PI3K-AKT and MEK-ERK signaling pathways in breast cancer. Further, this transmembrane mucin is also found to be overexpressed in ovarian cancer and various gastrointestinal malignancies including esophageal, colon and pancreatic cancers. Similarly, MUC4, another transmembrane mucin, is implicated in the pathobiology of cancers including pancreatic, breast, lung, and cervical cancers, in which MUC4 is involved in certain aspects of cancer metastasis, evasion of apoptosis, and induction of drug resistance. Overexpression of MUC1 represents a marker of aggressive biological behavior in non-small cell lung cancer, gastric and colorectal cancers. The relationship between MUC4 overexpression and tumor behavior is organ-dependent. For example, MUC4 overexpression is associated with more aggressiveness and increased metastases in breast cancer, extrahepatic bile duct carcinoma, and cholangiocarcinoma. Conversely, improved patient survival was associated with MUC4 expression in ovarian cancer, mucoepidermoid carcinoma of the salivary glands, and squamous cell carcinoma of the upper aerodigestive tract.

Mesothelin is a glycoprotein linked to cell surface via a glycosylphosphatidylinositol (Hassan et al., J Clin Oncol, 2016, 34(34):4171-9; Morello et al., Cancer Discov, 2016, 6(2):133-46; Einama et al., World J Gastrointest Pathophysiol, 2016, 7(2):218-22). Mesothelin is normally expressed at low level in mesothelial cells of the pleura, peritoneum and pericardium. Overexpression of mesothelin has been observed in many solid tumors, with particularly robust expression in mesothelioma, epithelial ovarian cancer, pancreatic adenocarcinoma, and extrahepatic biliary duct cancer. Aberrant mesothelin expression plays an active role in both malignant transformation of tumors and tumor aggressiveness by promoting cancer cell proliferation, contributing to local invasion and metastasis, and conferring resistance to apoptosis induced by cytotoxic agents. In addition, the high-affinity interaction between mesothelin and ovarian cancer antigen MUC 16 (cancer antigen 125) leads to heterotypic cell adhesion, which facilitates metastasis and increased resistance to anoikis.

Carbonic anhydrase is a family of metalloenzymes that catalyze the reversible hydration/dehydration of CO₂ to HCO₃⁻ and H⁺ in the presence of H₂O. The membrane associated isoforms IX and XII have been implicated in tumorigenicity, cancer metastasis, and as clinical prognosticators (Mboge et al., Metabolites, 2018, 8(1), pii:E19; Pastorek et at., Semin Cancer Biol, 2015, 31:52-64). Expression of carbonic anhydrase IX (CA IX) is modulated by hypoxia-inducible factor (HIF) in response to decreased oxygen levels and increased cell density. In contrast, carbonic anhydrase XII (CA XII) expression is robustly regulated by estrogen via estrogen receptor alpha (ERα). CA IX expression in normal tissues is restricted to stomach and epithelial tissues of the intestines and gallbladder. However it has been observed in many aggressive tumors including brain, breast, bladder, cervix, colorectum, head and neck, pancreas, kidney, lung, ovary, stomach, and B and T-cell lymphomas. Overexpression of CA IX in cancers is directly linked to many hypoxia- and acidosis-induced features of tumor phenotype including increased adaptation of tumor cells to microenvironmental stresses, resistance to therapy, increased tumor cell migration and invasiveness, increased focal adhesion during cell spreading, destabilization of intercellular contacts, maintenance of stem cell phenotype, tumor-stroma crosstalk, signal transduction and possibly other cancer-related phenomena. Expression of CA XII has been observed in many organs and at different developmental stages, and it is optimally active at higher pH values (pKa of ˜7.1) than CA XI (pKa of 6.3). High expression of CA XII has been observed in glioblastomas, astrocytomas, lung carcinomas, urinary bladder transitional cell carcinomas, ductal breast cancer, and T-cell lymphoma.

Cancer-testis antigens (CTAs) are considered as unique and promising cancer biomarkers and targets for cancer therapy (Gordeeva, Semin Cancer Biol, 2018, 53:75-89). Expression of CTAs in cancer cells is shown to result in their uncontrolled growth, resistance to cell death, potential to migrate, growth at distant sites (invasion and metastasis) and the ability to induce growth of new blood vessels (angiogenesis). The distinctive role of CTAs in carcinogenesis rests in their ability to stimulate a spontaneous immune response in cancer patients. CTA proteins are processed by the proteasome and some epitopes are presented by MHC class I molecules on the cancer cell surface. The frequency of CTA expression is highly variable depending on tumor types. The melanoma, liver, lung and ovarian cancers display a high frequency of CTA expression, breast, bladder, and prostate cancers display a moderate frequency of CTA expression, and hematopoietic, colon, renal and pancreatic cancers display a low frequency of CTA expression. Among 44 CTA gene families, melanoma antigen gene (MAGE) and New York esophageal squamous cell carcinoma 1 (NY-ESO-1) families are the most studied in cancer research. MAGEs can drive tumor progression through various mechanisms, which ultimately results in more aggressive, metastatic tumors that have greater chance of recurrence. They are also associated with enrichment in stem cell-like populations (Schooten et al., Cancer Treat Rev, 2018, 67:54-62; Weon et al., Curr Opin Cell Biol, 2015, 37:1-8). Broad expression of MAGEs is observed in cancers such as melanoma, brain, lung, prostate, and breast cancers. In addition, much higher expression of MAGEs is observed in cancer stem cell populations. For example, MAGE-A3 has much higher expression in a cancer stem cell-like side population in bladder cancer; MAGE-A2, -A3, -A4, -A6, -A12, and -B2 are highly enriched in the stem cell-like side population of multiple cancer cell lines. NY-ESO-1 expression has been reported in a wide range of tumor types. The most frequently expressed tumors include myxoid and round cell liposarcoma (89-100%), neuroblastoma (82%), synovial sarcoma (80%), melanoma (46%), and ovarian cancer (43%) (Thomas et al., Front Immunol, 2018, 9:947). For most cancer types, the expression of NY-ESO-1 is heterogeneous. However, myxoid and round cell liposarcomas shows expression in 94% of the cancer cells, and synovial sarcomas in 70%. Humoral and cellular immune response have been detected in a variety of cancer patients, including skin, colorectal, lung, breast, prostate, gastric, and hepatocellular cancers.

Gangliosides are a subfamily of glycosphingolipids that contain one or more sialic acid residues. Disialoganglioside with three glycosyl groups (GD3) and two glycosyl groups (GD2) have been characterized as oncofetal markers (Suzuki et al., Expert Opin Ther Targets, 2015, 19(3):349-62; Liang et al., Oncotarget, 2017, 8(29):47454-73; Fleurence et al., J Immunol Res, 2017, 2017:5604891). GD2 and GD3 are involved in embryonic development, and their expression is restricted to the central nervous system, predominantly in neuronal cell bodies, and mesenchymal stem cells, as well as peripheral nerves and skin melanocytes at low levels in healthy adults. GD2 is highly expressed on a variety of embryonic cancers (neuroblastoma, retinoblastoma, and rhabdomyosarcoma), bone tumors (osteosarcoma, and Ewing's sarcoma), soft tissue sarcomas (leiomyosarcoma, liposarcoma, and fibrosarcoma), neural crest derived tumors (small cell lung cancer and melanoma) and breast cancer. The expression of GD3 is upregulated in multiple tumors including melanoma and small cell lung cancer. GD2 and GD3 are associated with tumor cells proliferation, invasion and migration. GD2 and GD3 may also have distant effects on tumorigenesis and immunosuppression of human dendritic cells and T-cells. By interacting with different functional membrane proteins involved in cell adhesion and cell signaling in the glycolipid-enriched microdomain referred to as lipid rafts, GD2 and GD3 can regulate crucial cell functions such as cell proliferation, migration and resistance to chemotherapy. Of interest is the recent discovery of GD2 and GD3 in breast cancer stem-like cells, thought to contribute to tumor progression by self-renewal capacity and chemoresistance. Treatment of neuroblastoma with anti-GD2 antibodies has achieved a significant improvement in the survival of patients. However, severe side effect occurs including neuropathic pain, hypertension, and hematopoietic suppression. These side effects may be related to the possible immune recognition of GD2 and/or cross-reaction with its epitope neighbors in its synthesis pathway, such as GD1b and GD3, on sensitive nerve fibers and on mesenchymal stromal cells in the marrow microenvironment.

Almost all cancers overexpress one or more cell surface markers in cancer cells and/or non-cancer cells in the tumor microenvironment. Selectively targeting these overexpressed cell surface markers without off-tumor interaction will substantially improve the efficacy and expand the treatment options in cancer therapy. Also, more of these markers could be used for cancer diagnosis and prognosis with better accuracy. However, the current cancer targeting therapeutics, such as antibodies, can barely discriminate between the overexpressed targets in tumors and normally-expressed ones in healthy tissues, and cause a variety of side effects. Associated with this limitation, so far only a small number of these targets have been successfully targeted in cancer therapy. By applying the principles of multivalency, this invention provides multivalent pharmacophores that can achieve selectivity to the overexpressed cell surface markers in cancers without or with much reduced binding to normally-expressed targets in healthy tissues. At the same time, higher avidity than the affinity of antibodies can be obtained toward the overexpressed targets. In addition, the pharmacophores will not be interfered by high concentration of cleaved and soluble targets present in the circulatory systems and in tumor microenvironment. Furthermore, by clustering the cell surface markers, the multivalent pharmacophore has the potential to induce or inhibit endocytosis of the bound targets and thus to affect cell growth or death, or to disrupt the functions of bound cell surface markers. Compared to therapeutic antibodies, the multivalent pharmacophores can be synthesized with lower molecular weight and thus better tissue penetration property. Owing to these unique properties, the multivalent pharmacophores as disclosed in this invention form a different class of targeting therapeutics and diagnostics that can provide better efficacy in cancer treatment and higher accuracy in cancer diagnosis than the currently available therapeutic and diagnostic means. In addition, the multivalent pharmacophores as described in this invention will dramatically expand the range of targets that can be targeted in both cancer treatment and diagnosis, and more types of cancers will be treated target-specifically as a result. Besides cancers, the present invention can also target the overexpressed cell surface markers in other diseases including infectious diseases.

### 2. Definitions

It must be noted that, as used herein, the singular forms “a”, “an”, and “the” includes plural referents unless the context clearly dictates otherwise. Likewise, the plural terms shall also include the singularity unless otherwise required by the context.

As used herein, “comprising”, “including”, “consisting”, and grammatical equivalents thereof are inclusive or open-ended terms that do not exclude additional and unrecited elements, methods or steps.

The use of the term “or” in the claims is used to mean “and/or” unless explicitly indicated to refer to alternatives only or the alternatives are mutually exclusive, although the disclosure supports a definition that refers to only alternatives and “and/or”.

The term “overexpression” or “overexpressed” is defined by method of immunohistochemistry, immunofluorescence, flow cytometry, or other methods of quantification in which the quantity of elements measured in cancers or other diseased cells/tissues is at least 2-fold of that in normal cells/tissues, even though the difference can be 10-fold or higher.

The term “pharmacophore” refers to a biological molecule that is capable of making a biological or pharmaceutical interaction with a specific target with or without triggering biological responses.

The term “ectodomain” refers to extracellular domain of a membrane protein.

The term “monomer” refers to a ligand that is able to bind and form a monovalent bond with its cognate site.

The term “ligand” refers to a biomolecule that is able to bind to a target or receptor to form a complex with or without biological consequences.

The term “natural ligand” refers to the ligand that is made by human cells and other living organism, whereas “synthetic ligand” refers to the ligand that does not exist in humans or other natural living organisms and instead is created through genetic engineering process or by chemical synthesis. Natural ligand can be synthesized chemically, but its structure is similar to naturally produced one.

The term “receptor” refers to a biomolecule that can be specifically bound by a ligand to form a complex with or without biological consequences.

The terms “cell surface target”, “cell surface marker” and “cell surface receptor” are used interchangeably, and refer to molecules on cell surface that can be recognized specifically by a ligand and form a bond.

The term “affinity” refers to the strength of a single bond between a ligand and its target, whereas “avidity”, also known as functional affinity, refers to the accumulated strength of multiple coordinated interactions between multivalent ligands and their cognate targets. Although common antibody, such as IgG, forms double bonds with its targets, affinity is usually used to describe its binding strength. Affinity and avidity can be quantified by an association rate constant (Kₐ) and a dissociation rate constant (K_{d}) at equilibrium. K_{d} value and binding affinity or avidity are inversely related.

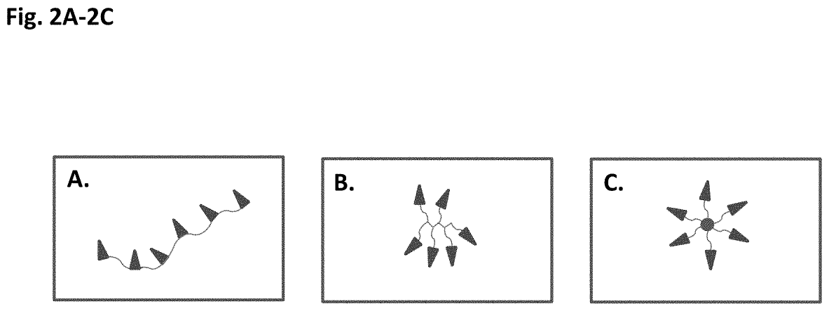

The term “branched linker” refers to a linker consisting of at least 3 branches extending from one stem (an example shown in FIG. 2B), while “star-shaped linker” refers to a linker consisting of at least 3 branches extending from one central core or nodule (an example shown in FIG. 2C). The linker links all the ligands together to make a multivalent pharmacophore.

### 3. Detailed Description of the Embodiments

While the description sets forth various embodiments with specific details, it will be appreciated that the description is illustrative only and should not be construed in any way as limiting the invention. Furthermore, various applications of the inventive concepts and modification thereof, which may occur to those who are skilled in the art, are also encompassed by the general concepts described below. Also, it is to be understood that the phraseology and terminology employed herein is for the purpose of description and should not be regarded as limiting.

The present invention provides a multivalent pharmacophore for treating and diagnosing cancers or other diseases by specifically binding to the cell surface markers overexpressed in cancers or other diseased cells with high avidity. The multivalent pharmacophore is synthesized by linking multiple targeting monomers or ligands to the branches of branched linker or star-shaped linker. In one particular embodiment, the present invention describes a multivalent pharmacophore that uses PD-1 ectodomain as linked monomers or ligands and specifically blocks immune checkpoint signalings involving the axes of PD-1/PD-L1, PD-1/PD-L2, and CD80/PD-L1. In another particular embodiment, the present invention describes a multivalent pharmacophore that uses SIRPα IgV-like domain as linked monomers or ligands for targeting cancer cells overexpressing CD47 and blocks the related checkpoint signaling. In yet another particular embodiment, the present invention describes a multivalent pharmacophore that uses N-terminal growth factor-like domain (GFD) of uPA as linked monomers or ligands for competitive binding to overexpressed uPAR in cancers and inhibits the functions of urokinase-type plasminogen activator system.

In some embodiments, a multivalent pharmacophore uses ectodomains as ligands. These ectodomains, such as those of PD-1, SIRPα, ICAM and VCAM-1, can bind to their natural cognate targets, receptors, or ligands on cell membrane.

In other embodiments, natural ligands are used as ligands of multivalent pharmacophore for binding to their cognate receptors on cell membrane. The natural ligands can be enzymes or zymogens secreted from cells, such as matrix metallopeptidases (MMPs), uPA and pro-uPA. The natural ligands can also be hormones, cytokines, chemokines, or other signaling molecules, such as VEGF, FGF2, TGFβ, PDGF and transferrin. Further, the natural ligands can be components of extracellular matrix that bind to cellular receptors, including cell adhesion proteins such as fibrinogen, fibronectin, collagen, laminin and fibrin, proteoglycans such as heparan sulfate and chondroitin sulfate, and non-proteoglycan polysaccharides such as hyaluronan. The natural ligands can also be molecules absorbed as nutrients such as folate. In addition, the natural ligands can be glycan containing molecules such as polysaccharides, glycosaminoglycans, glycoproteins, proteoglycans, or glycolipids that bind to carbohydrate binding cell surface proteins.

In some embodiments, synthetic molecules are used as ligands of multivalent pharmacophore. The synthetic ligands do not exist in humans or other natural living organisms but are created by genetic engineering process or by chemical synthesis including, for example, a single chain variable fragment (scFv), single-domain antibody, affimer, aptamer, peptide, cyclic peptide, D-peptide, or chemical compound.

In case of ectodomain used as ligands of the multivalent pharmacophores, the ectodomain has the full length sequence of the ectodomain in some embodiments; it can also be a fragment or truncated version of the ectodomain in other embodiments. In some embodiments, the ectodomain possesses the native polypeptide sequences, whereas in other embodiments, the ectodomain has one or more amino acids mutated.

In situations where natural ligands that are polypeptides are used as ligands of the multivalent pharmacophores, the natural ligand has the full length sequence of the polypeptide in some embodiments, whereas it is only a fragment or truncated version of the natural ligand in other embodiments. In certain embodiments, the natural ligand possesses the native polypeptide sequence, whereas in alternative embodiments, the natural ligand has one or more amino acids mutated.

In situations where natural ligands that contain polysaccharides are used as ligands of the multivalent pharmacophores to target overexpressed carbohydrate-binding cell surface proteins, the natural ligand has the full length sequence of the polysaccharide in some embodiments, whereas it is only a fragment or truncated version of the polysaccharide in other embodiments. In some embodiments, the natural ligand possesses the native polysaccharide sequence, whereas in different embodiments, the natural ligand has one or more sugar units changed.

To limit monovalent binding between multivalent pharmacophores and one of their cognate receptors, in some embodiments, the ligand that is made of ectodomain or natural ligand has the same binding affinity as the endogenous counterpart, or up to 100-fold lower affinity than the endogenous counterpart. In different embodiments, the ligand that is made of ectodomain or natural ligand can have the binding affinity up to 5-fold higher than the endogenous counterpart if the binding between the multivalent pharmacophores and normally-expressed targets is less often than the binding between the endogenous counterpart and normally-expressed targets.

In some embodiments, when the ligands of a multivalent pharmacophore are synthetic ligands, the binding affinity of the synthetic ligand to its target is limited to low to moderate levels. The low to moderate affinity is defined as the dissociation constant (K_{d}) in the range of 0.01 μM and 10 μM.

With these affinity limitation strategies, monovalent binding between the multivalent pharmacophore and one of its cognate receptors usually does not occur, and thus the pharmacophore will rarely binds to normally-expressed targets and no substantial side effects associated with off-tumor binding will happen. Of note, the actually chosen affinity for ligands of a particular pharmacophore will be target specific, with some targets allow ligands with higher affinity than the others.

There are many cell surface proteins and cell membrane-associated non-protein components overexpressed in cancer cells and/or non-cancer cells in the tumor microenvironment or, in the case of infectious diseases, in immune cells and infected cells. They all can be targeted by the multivalent pharmacophore described in the present invention. In some embodiments, these cell surface markers are overexpressed PD-L1, PD-L2, PD-1, B7-H3, B7x, B7-H4, galectins, TIM-3, CD74, CD47, or CD24. In other embodiments, they are overexpressed CXCR4, folate receptor, or transferrin receptor (TfR). In yet some other embodiments, they are overexpressed EGFR, EGFRvIII, HER2, HER3, HER4, PDGFRα and β, FGFRs, ALK, EphA2, or insulin-like growth factor receptors (IGF-1R and INSR-A). In further embodiments, they are overexpressed ATP-binding cassette (ABC) transporters (P-gp, BCRP and MRP1), claudins, EpCAM, carcinoembryonic antigen-related cell adhesion molecules (CEA and CEACAM6), CD44, or integrins. In different embodiments, they are overexpressed urokinase-type plasminogen activator receptor (uPAR), type II transmembrane serine proteases (matriptase, hepsin and TMPRSS2), proteoglycans (CSPG4, glypicans and syndecans), mucins, mesothelin, carbonic anhydrase IX and XII, cancer-testis antigens (MAGEs and NY-ESO-1), or gangliosides (GD2 and GD3). These overexpressed cell surface markers can be specifically targeted with high avidity by multivalent pharmacophores linked with ectodomains, natural ligands, or synthetic ligands.

In some embodiments, the multivalent pharmacophore binds to the same site of the cell surface markers as the endogenous ectodomains and natural ligands, and acts as a competitor to them. Competitive binding of the pharmacophore to these overexpressed markers can induce or decrease, block, inhibit, abrogate and interfere with signal transduction associated with the overexpressed markers. In other embodiments, the multivalent pharmacophore binds to non-competitive binding site of the markers with or without inducing inhibitory or stimulatory activities in the targeted cells.

Besides interfering with target-associated signaling pathways, the multivalent pharmacophores can also induce other functional activities by clustering the bound cell surface markers, including induction or inhibition of surface marker endocytosis, disruption of the functions of the bound markers, induction of conformational changes of the targeted markers and inhibition of cell growth or induction of cell death.

In certain embodiments, a multivalent pharmacophore is composed of ligands that recognize the same type of targets (mono-specificity). In other embodiments, the ligands recognize more than one type of targets (multi-specificity). For example, if the cancer cells overexpress both PD-L1 and CD47, a multivalent pharmacophore that blocks both targets may have better efficacy.

In some embodiments, the multivalent pharmacophore is synthesized using branched linkers. In other embodiments, the multivalent pharmacophores is synthesized using star-shaped linkers. The branched and star-shaped linkers have 3 branches, or 4, 5, 6, 7, 8, 9 and 10 branches for each linker. Although linkers with even higher number of branches can be synthesized, those with up to 10 branches usually will provide high enough avidity and specificity for therapeutic and diagnostic purposes. The ligands or monomers of multivalent pharmacophore are conjugated to the free end of the branches. In all embodiments, the branched linker or star-shaped linker has branches extending or radiating from one common stem or central core, such that all of the linked monomers or ligands are grouped in a form of cluster and are close to each other. In addition, the distance between the ligands is not varied as widely as would see in a linear linker, and neither is the valency fluctuated as much as in a linear linker. In comparison to linear linker, the arrangement of branches and the linked ligands as proposed in this invention can achieve higher effective concentration and sharper discrimination between overexpressed targets and normally-expressed ones.

The effective concentration is also influenced by the length of the branches. Generally, the shorter the branches are, the closer the ligands become and the higher effective concentration the pharmacophore will have. Besides, long branches are easier to get entangled among themselves. However, the branches need to be long enough so that multivalent interaction can take place, and also have enough length for the linked ligands to orient themselves freely and optimally interact with the targets. More importantly, for the purpose of target density discrimination between overexpressed targets and normally-expressed ones, the length of the branches is chosen in such a way so that the pharmacophores can have multivalent interactions with the overexpressed but not with normally-expressed targets. Therefore, in some embodiments, the length of the branches is 2 nm, whereas in other embodiments the branches can be up to 60 nm long, depending on the density of the overexpressed targets and the freedom and accessibility of the linked ligands to their targets. However, in all embodiments, shorter branches will be preferred in order to achieve higher avidity and selectivity, and decrease the chance of entanglement among themselves.

The flexibility of linkers and branches also influences effective concentration. Rigid branches and linkers/scaffold with limited flexibility would restrict the ligands' orientation and effective interactions with cognate receptors. Flexible structure of the multivalent pharmacophore can allow ligands to adopt a variety of conformations and orientations to effectively bind the targets with low steric strains. Therefore, the branches, linker or scaffold of the pharmacophore in this invention is preferably made of flexible molecules. In particular embodiments, the branches and linkers are made of poly(ethylene glycol) (PEG), poly(N-vinylpyrrolidone) (PVP), polyglycerol (PG), poly(N-(2-hydroxypropyl) methacrylamide) (PHPMA), polyoxazolines (POZs), polysaccharides, poly(amino acid), or any combination of these materials.

The multivalent pharmacophore of the present invention can be used as a carrier to bring a variety of therapeutic agents or detectable labels to the cells overexpressing cognate targets. In some embodiments, the pharmacophore is coupled to or conjugated with chemotherapeutic drugs. In other embodiments, the pharmacophore is coupled to or conjugated with cytotoxic or cytostatic agents. Such agents can be ricin, abrin, diphtheria toxin, emtansine (DM1), chalicheamicins, monomethyl auristatin E, or the like. In some embodiments, the pharmacophore is coupled to or conjugated with radionuclides, such as yttrium-90, indium-111, or the like. In additional embodiments, the pharmacophore is coupled to or conjugated with immunologic adjuvants, such as Toll-like receptor agonists. In other embodiments, the pharmacophore is coupled to or conjugated with immune effector. This can be, for example, part or whole of immunoglobulin constant domain (Fc) for binding to Fc receptor expressed in immune cells, or 4-1BB agonist, OX40 ligand and CD40 ligand for immune stimulation, or CD3 ligand to link T-cells to tumor cells. In further embodiments, the pharmacophore is coupled to or conjugated with cytokines, such as IL-2 and IL-12 to enhance immune response, or IL-10 for reduction of inflammation. In different embodiments, the pharmacophore is coupled to or conjugated with detectable labels that can be used, e.g., for imaging or diagnosis. Non-limiting examples of detectable labels include radiography moieties, e.g., heavy metals and radiation emitting moieties, positron emitting moieties, magnetic resonance contrast moieties, and optically visible particles. It will be appreciated by one of ordinary skill that some overlap exists between what is a therapeutic moiety and what is an imaging moiety.

In some embodiments, one type of agent is coupled to or conjugated with a multivalent pharmacophore, while in other embodiments, more than one type of agents is coupled to or conjugated with the pharmacophore. In certain embodiments, the agent is coupled to or conjugated with ligands of the multivalent pharmacophore. In other embodiments, the agent is coupled to or conjugated with the linker or scaffold of the pharmacophore. In yet some other embodiments, the agents are coupled to or conjugated with both ligands and linkers/scaffold of the pharmacophore.

In some embodiments, the multivalent pharmacophore is used as a high avidity competitor against high affinity endogenous ligands for the overexpressed cognate markers, such as the high affinity interaction between uPA/pro-uPA and uPAR. In this specific case, competitive antibodies or other similar agents do not have high enough affinity for efficient competition, especially when the endogenous ligands are also overexpressed in the tumor microenvironment

In some embodiments, the multivalent pharmacophore is used as a therapeutic agent, whereas in other embodiments, it is used as a diagnostic or prognostic agent. In some embodiments, the multivalent pharmacophore can be used as an adjuvant therapy, or combination with other therapy. In addition to be used as a therapeutic, prognostic, or diagnostic agent, in some embodiments, the multivalent pharmacophore can be used as a disease preventive or treatment maintenance agent.

In some embodiments, the multivalent pharmacophore can be used for treatment and diagnosis of cancer patients. In other embodiments, the multivalent pharmacophore can be used as a therapeutic/diagnostic agent for chronic viral, bacterial, or parasitic infectious diseases that overexpress cell surface markers in the diseased cells and immune cells.

### 4. Examples

The following examples are putting forth to illustrate, but not to limit, the scope of the claimed invention, nor are they intended to represent that the experiments and applications below are all or the only experiments and applications performed or could be performed.

### Example 1: Overexpressed-Target Specific and Combined PD-1/PD-L1/PD-L2 and PD-L1/CD80 Axes Immune Checkpoint Blockade with High Avidity Tetravalent/Hexavalent PEG PD-1 Ectodomain Pharmacophores (4-Arm PEG PD-1_{ecto} and 6-Arm PEG PD-1_{ecto})

### Background

Cancer immunotherapy with antibodies to PD-1, PD-L1 and CTLA-4 has achieved impressive success. So far, two anti-PD-1 and three anti-PD-L1 antibodies are in clinical use for the treatment of multiple types of cancers, including melanoma, non-small cell lung cancer, renal cell carcinoma, head and neck squamous cell carcinoma, urothelial carcinoma, Merkel cell carcinoma, Hodgkin's lymphoma, microsatellite instability high and mismatch repair deficient colorectal cancer or other solid tumors, hepatocellular carcinoma, and gastric cancer. More agents targeting PD-1 and PD-L1 are in different stage of development (Tang et al., Ann Oncol, 2018, 29(1):84-91).

Nevertheless, a sobering reality about current immune checkpoint therapies is their low response rate: only a proportion of patients show objective responses to the treatments, with only a small fraction experiencing complete responses. This is likely due to the complex network of immunosuppressive pathways present in advanced tumors, which are unlikely overcome by the blockage at a single checkpoint. In fact, many PD-L1 positive tumors do not respond to the treatment of anti-PD-1 or anti-PD-L1 antibodies, whereas some PD-L1 negative tumors do response (Sun et al., Immunity, 2018, 48(3):434-52). PD-1 is an immune checkpoint receptor and expressed mainly on the surface of activated T cells, B cells, and monocytes/macrophages. Anti-PD-1 antibody inhibits the checkpoint signaling by preventing the engagement of PD-1 with its ligands, PD-L1 and PD-L2. PD-L1 is overexpressed in a variety of cancer cells and cancer stromal cells, both contributing to immune suppression in a non-redundant fashion (Lau et al., Nat Commun, 2017, 8:14572; Herbst et al., Nature, 2014, 515(7528):563-7). Besides functioning as the ligand for PD-1, PD-L1 also engages CD80 to deliver bidirectional inhibitory signals to activated T cells (Butte et al., Immunity, 2007, 27(1):111-22; Park et al., Blood, 2010, 116(8):1291-8; Paterson et al., J Immunol, 2011, 187(3):1097-105). Anti-PD-L1 antibody, therefore, inhibits the checkpoint signaling by prohibiting the interaction of PD-L1 with PD-1 and CD80. Consequently, neither anti-PD-1 nor anti-PD-L1 antibody alone can block all the checkpoint signaling that involves PD-1/PD-L1, PD-1/PD-L2, and CD80/PD-L1 axes. In the case of anti-PD-1 antibody treatment, CD80/PD-L1 signaling is still intact and T cell activation can be compromised as a result. Likewise, anti-PD-L1 antibody fails to prevent interaction of PD-1 with PD-L2. It is generally believed that PD-L2 and CD80 do not have much chances engaging PD-1 and PD-L1, respectively, mainly because of their low expression levels in normal situations, and thus have limited impact on immune checkpoint (Li et al., J Biol Chem, 2017, 292(16):6799-809; Cheng et al., J Biol Chem, 2013, 288(17):11771-85). However, when PD-1 or PD-L1 is blocked by respective antibody, the possibility for PD-L1 to engage CD80 or for PD-L2 to engage PD-1 will increase accordingly. More importantly, PD-L2 has been found overexpressed in many tumor types and present in stromal, tumor, and endothelial cells (Yearlet et al., Clin Cancer Res, 2017, 23(12):3158-67; Jung et al., Cancer Res Teat, 2017, 49(1):246-54; Shin et al., Ann Surg Oncol, 2016, 23(2):694-702; Baptista et al., Hum Pathol, 2016, 47(1):78-84; Calles et al., J Thorac Oncol, 2015, 10(12):1726-35). As reviewed by Sun, et al. (Immunity, 2018, 48(3):434-52), co-amplification of PD-L1 and PD-L2 in different types of tumors are observed and, interestingly, exposure to type I interferons has a much greater effect on PD-L2 than on PD-L1 expression in melanoma cells. It has also been reported that both PD-L1 and PD-L2 positivity on combined tumor, stromal and immune cells significantly predicted clinical response to pembrolizumab, an anti-PD-1 antibody, with PD-L2's prediction independent of PD-L1's (Yearly et al., Clin Cancer Res, 2017, 23(12):3158-67). There are few reports on CD80 expression in tumor microenvironment so far. However, CD80 expression could be induced in situations of activated immune response (Park et al., Blood, 2010, 116(8):1291-8) or by interferon α/γ (Wan et al., J Immunol, 2006, 177(12):8844-50). Therefore, an increased expression of CD80 in the tumor microenvironment can be expected if there is an active T-cell immune response in those tumors. Taken together, it is likely that a better efficacy can be achieved for the treatment that blocks all interactions involving PD-1/PD-L1, PD-1/PD-L2, and PD-L1/CD80 axes, especially for cancers expressing high levels of PD-L2 and/or CD80 in addition to PD-1 and PD-L1. To be noted, the affinity between human PD-1 and PD-L2 is 3-4 folds higher than that between PD-1 and PD-L1, whereas the affinity between human CD80 and PD-L1 is 2-4 folds weaker than that between PD-1 and PD-L1 (Cheng et al., J Biol Chem, 2013, 288(17):11771-85).

An extracellular domain or ectodomain of PD-1, which is a soluble form of PD-1, can interact with both PD-L1 and PD-L2 and prevent their engagement with cell membrane-associated PD-1. In addition, binding of soluble PD-1 to PD-L1 also prohibits interaction of PD-L1 with CD80 because both PD-1 and CD80 have overlapping binding site on PD-L1 (Butte et al., Immunity, 2007, 27(1):111-22). Therefore, a soluble PD-1 possesses the functions equivalent to the combined actions of anti-PD-1 and anti-PD-L1 antibodies. Additionally, checkpoint blockade with soluble PD-1 can overcome potential targeted therapy-driven mutations and off-target escape (Tan et al., Protein Cell, 2018, 7(12):866-77; Russo et al., Science, 2019, 366(6472):1473-80), because any mutation of PD-L1 and PD-L2 that fails to bind the soluble PD-1 will not engage cell membrane associated PD-1. Experiment showed that the soluble PD-1 blocked the inhibitory signaling to T-cells by PD-L1 on surface of stromal cells and promoted T cell proliferation, which may be responsible for the persistent activation of self-reactive T-cells (Wan et al., J Immunol, 2006, 177(12):8844-50). However, the affinity between PD-1 and PD-L1 or PD-L2 is low, with Kd values of ˜8 μM and ˜2 μM, respectively, as measured by Surface Plasmon Resonance, or 1.6 μM and 0.5 μM, respectively, as measured by isothermal titration calorimetry (Cheng et al., J Biol Chem, 2013, 288(17):11771-85). It is impossible to deliver sufficient amount of soluble PD-1 in cancer patients to reach the concentration high enough to compete against cell bound PD-1 which is usually also overexpressed on activated T cells in tumor microenvironment.

As described previously, multivalent interactions can achieve high avidity and specificity to overexpressed targets. In this invention, tetravalent and hexavalent PEG PD-1 ectodomain pharmacophores (4-arm PEG PD-1_{ecto} and 6-arm PEG PD-1_{ecto}) are disclosed which are synthesized by conjugating multiple copies of PD-1 ectodomain (PD-1_{ecto}) site-specifically onto a tetravalent or hexavalent polyethylene glycol homopolymer (4-arm PEG or 6-arm PEG) to form 4-arm PEG PD-1_{ecto} and 6-arm PEG PD-1_{ecto}. Because PD-L1 and/or PD-L2 are overexpressed on the surface of cancer cells and cancer stromal cells, 4-arm PEG PD-1_{ecto} and 6-arm PEG PD-1_{ecto} as described in this invention will selectively bind to those cells with high avidity and inhibit the related immune checkpoints. At the same time, side-effects caused by off-target and/or on-target/off-tumor binding will be limited. Especially, the cancer patients with autoimmune diseases can be treated with much fewer risks of toxicity from general inhibition of the immune checkpoints. Furthermore, as described before, the multivalent PEG PD-1_{ecto} pharmacophores will not be interfered by the soluble PD-L1 which is usual high in the blood and lymphatic circulatory systems and in tumor microenvironment of PD-L1 overexpressing cancer patients. Therefore, 4-arm PEG PD-1_{ecto} and 6-arm PEG PD-1_{ecto} described in this invention can offer better efficacy with fewer side effects than the antagonistic anti-PD-1/PD-L1 antibodies that are in use today. In addition to acting as a linker for PD-1_{ecto}, 4-arm PEG and 6-arm PEG can improve the solubility and stability of the linked proteins/polypeptides due to the characteristics associated with PEGylation (Turecek et al., J Pharm Sci, 2016, 105(2):460-75).

### Experimental Results:

(1) Production of PD-1_{ecto}-Mycobacterium xenopi GyrA Intein Fusion Protein and PD-1_{ecto} Hydrazide.

A method of expressed protein ligation (EPL) is used to conjugate C-terminus of PD-1_{ecto} site-specifically to the arms of 4-arm PEG or 6-arm PEG, as adapted from Thom et al. (Bioconjug Chem, 2011, 22(6):1017-20).

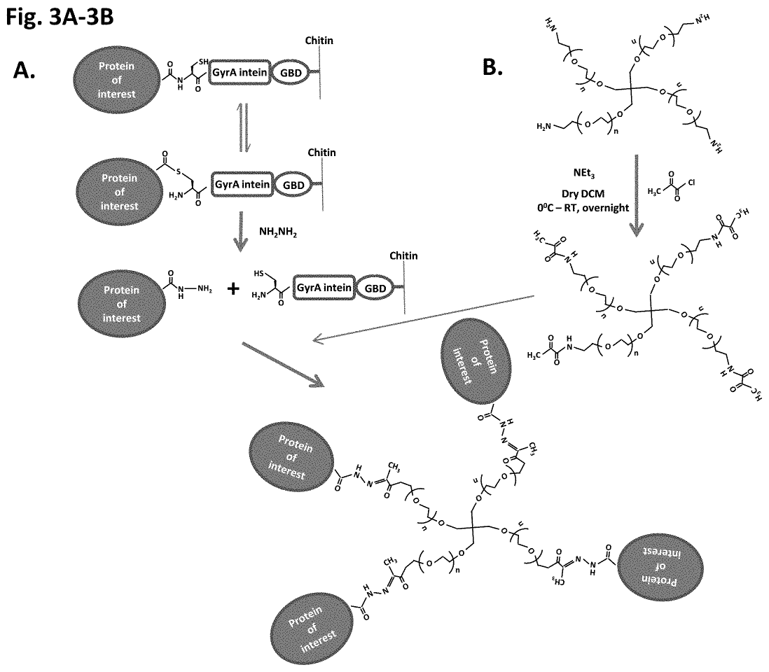

The sequence of PD-1_{ecto} (aa. 21-169) is cloned into IMPACT vector (pTXB1, New England BioLabs) as a C-terminal fusion protein. This pTXB1 PD-1_{ecto} construct encodes the 149 amino acids of PD-1 ectodomain which is linked via leucine to the N-terminus of Mycobacterium xenopi GyrA intein that is in turn fused to the N-terminus of a chitin binding domain (CBD) (FIG. 3A). The construct is transformed into E. coli strain T7 Express (New England BioLabs). A freshly grown colony is inoculated into LB medium containing 100 μg/mL ampicillin and the cells grow at 37° C. When the OD₆₀₀ of the culture reaches 0.5-0.7, protein expression is induced by adding isopropyl β-D-thiogalactopyranoside (IPTG) to a final concentration of 0.3 mM and the culture is incubated overnight at 18° C. The cells are pelleted by centrifugation at 5000 g for 20 min at 4° C., and lysed in lysis buffer (20 mM sodium phosphate pH 7.4, 0.5 M NaCl, 0.5 mM EDTA, 15% glycerol, 0.1% Sarkosyl NL) with 1 mM AEBSF by sonication. The soluble fraction is mixed with chitin beads pre-equilibrated in lysis buffer, 4° C. for 1.5 h. The beads are then washed extensively with lysis buffer followed by cleavage buffer (200 mM sodium phosphate pH 7.4, 200 mM NaCl, 0.05% Zwittergent 3-14) to yield purified PD-1_{ecto} GyrA intein CBD fusion protein immobilized on chitin beads. These beads are mixed with 1% hydrazine in cleavage buffer with 1 mM EDTA at room temperature overnight. The soluble supernatant contains the cleaved PD-1_{ecto} hydrazide, which is purified by RP HPLC on a Gilson preparative HPLC system with a Jupiter C5 column (Phenomenex) using a 60 min linear gradient, 10-100% B at a flow rate of 2 mL/min. Buffer A: 0.1% TFA/H₂O (V/V) and buffer B: 0.1% TFA/60% acetonitrile/40% H₂O (V/V/V). Fractions are analyzed by electrospray mass spectrometry and analytic RP HPLC. Pure fractions are pooled and lyophilized. The sequence of PD-1_{ecto} hydrazide made here is:

PGWFLDSPDRPWNPPTFSPALLVVTEGDNATFTCSFSNTSESFVLNWYR
MSPSNQTDKLAAFPEDRSQPGQDCRFRVTQLPNGRDFHMSVVRARRNDS
GTYLCGAISLAPKAQIKESLRAELRVTERRAEVPTAHPSPSPRPAGQFQ
TL-NHNH₂

(2) Generation of Pyruvoyl 4-Arm PEG and Pyruvoyl 6-Arm PEG.

4-arm and 6-arm PEG PD-1_{ecto} pharmacophores with different arm lengths are synthesized for the purpose of testing conjugation efficiency and evaluating the target binding specificity for the different types of multivalent pharmacophores.

Four types of multi-arm homofunctional PEG are obtained from Ruixibiotech. 4-arm PEG-2K amine has an average of 11 units of ethylene oxide (EO) for each arm, and 4-arm PEG-6K amine has an average of 33 units of EO for each arm, which corresponds to an estimated contour length of 3 and 9 nm, respectively. 6-arm PEG-3.4K amine has an average of 12 units of EO for each arm, and 6-arm PEG-10K amine has an average of 37 units of EO for each arm, which corresponds to an estimated contour length of 3.3 and 10 nm, respectively. A 4-arm PEG-amine is shown for example in FIG. 3B.

First, pyruvoyl chloride is formed by treatment of pyruvic acid with α,α-dichloromethyl methyl ether. The pyruvoyl 4-arm PEG and 6-arm PEG are formed by overnight coupling between pyruvoyl chloride and 4-arm PEG-amine or 6-arm PEG-amine (FIG. 3B). Briefly, the PEG-amine (500 mg, 50 mmol) in anhydrous DCM (5 mL) is treated with pyruvoyl chloride (10 mg, 100 mmol) under N₂ in the presence of triethylamine (11 μL, 100 nmol) at 0° C. The reaction mixture is allowed to warm to room temperature and stirred overnight. Aqueous workup and trituration with Et₂O afforded the pure product as a white solid.

(3) Synthesis of 4-Arm PEG PD-1_{ecto} and 6-Arm PEG PD-1_{ecto} Pharmacophores by Site-Specific C-Terminal PEGylation with Intein-Mediated Protein Ligation.

PD-1_{ecto} C-terminal hydrazide is dissolved in 100 μL 40% acetonitrile with 0.1% TFA to a final concentration of ˜250 μM. A 20 fold molar excess of pyruvoyl 4-arm PEG or 6-arm PEG is added and reactions left at room temperature overnight (˜16 hours) (FIG. 3). The PEGylation reaction is diluted 10 fold in buffer A (20 mM Tris-HCl pH7.3, 0.05% Zwittergent 3-14) and loaded onto a 1 mM HiTap Q FF anion exchange column via AKTA purified system (GE Healthcare). The column is washed with 5-10 CV buffer A to remove the unbound, unreacted pyruvoyl-PEG and the bound protein is eluted over a 0.1 M NaCl gradient (20 CV). Unbound and eluted fractions are analyzed on duplicate SDS PAGE gels, one is stained with Coomassie blue and the other stained for PEG. The fractions containing the desired 4-arm PEG PD-1_{ecto} or 6-arm PEG PD-1_{ecto} are concentrated using VivaSpin2 centrifugal concentrators (Sartorius) and run through a Superdex 200 10/300 GL column (GE Health) on 10 mM sodium phosphate pH7.4, 50 mM NaCl, 0.05% Zwittergent 3-14. This yields pure site-specifically C-terminal PEGylated multivalent PD-1_{ecto} pharmacophores.

(4) Specific Binding of 4-Arm PEG PD-1_{ecto} and 6-Arm PEG PD-1_{ecto} to PD-L1 and PD-L2 Coated Plates.

Human recombinant PD-L1, PD-L2 and CD80 (Abcam) at the concentration of 40 nM were coated onto wells of 96-well MSD plate (small spot, 40 μL/well) overnight at 4° C., and then blocked with 3% MSD Blocking Buffer for 1 h. After washing the plate with MSD Wash Buffer, recombinant human PD-1 Fc chimera protein and PD-L1 Fc chimera protein (R&D Systems), 4-arm PEG-2K PD-1_{ecto}, 4-arm PEG-6K PD-1_{ecto}, 6-arm PEG-3.4K PD-1_{ecto}, and 6-arm PEG-10K PD-1_{ecto}, all at 5 nM, were diluted in 1% MSD Blocking Buffer and added into wells at 40 μL/well. After shaking the plate at room temperature for 2 h and washing, mouse monoclonal anti-human PD-1 antibody (Thermo Fisher Scientific) was added into 4-arm/6-arm PEG PD-1_{ecto} and PD-1 Fc chimera protein treated wells. The plates were shaken for another 1 h at room temperature. After washing, SULFO-TAG anti-mouse IgG antibody was added into the plates. The plates were further incubated for 1 h and then washed and read after adding 1×MSD Read Buffer. SULFO-TAG anti-human IgG antibody was added into the plate of PD-L1 Fc chimera proteins. The wells without coating were used as background binding.

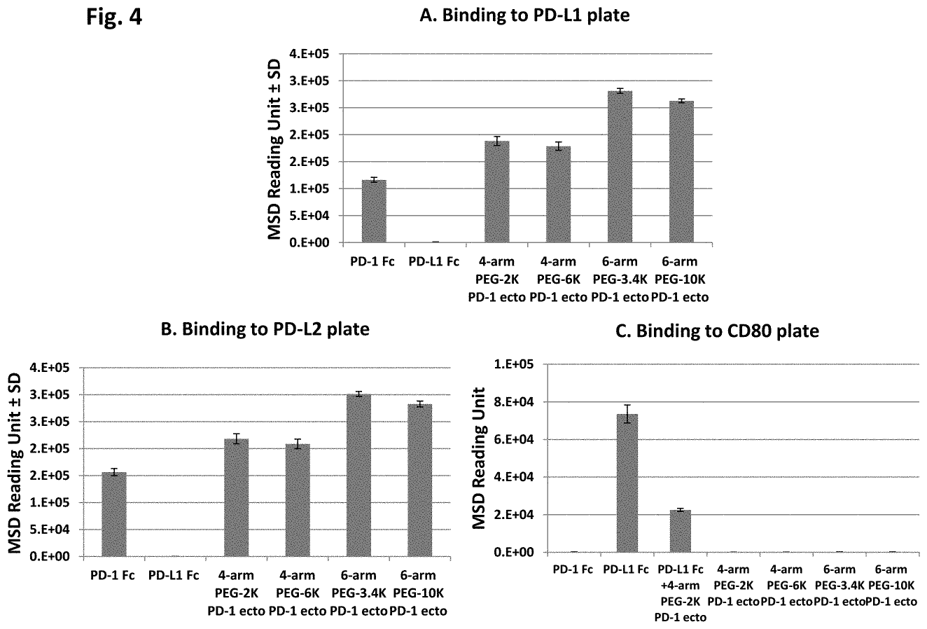

As shown in FIGS. 4A and B, 4-arm PEG PD-1_{ecto} and 6-arm PEG PD-1_{ecto}, as well as PD-1 Fc chimera bound to PD-L1 and PD-L2 plates, with 6-arm PEG PD-1_{ecto} showing the highest binding likely due to more units of PD-1 in each pharmacophore and higher binding avidity. The binding to CD80 plate was observed only for PD-L1 Fc chimera, which was substantially inhibited by the presence of 4-arm PEG PD-1_{ecto} because of competitive binding against CD80 to the overlapping binding site on PD-L1 (FIG. 3C). These results confirmed the binding capability and selectivity for the synthesized multivalent PEG PD-1_{ecto} pharmacophores.

(5) Selective Binding of 4-Arm PEG PD-1_{ecto} and 6-Arm PEG PD-1_{ecto} to High-Density PD-L1 Coated Plates.

Human recombinant PD-L1 was coated onto wells of MSD 96-well plate with the concentrations starting from 80 nM and 1:1 dilution for 8 titrations (40 μL/well). The empty wells were added with PBS. After overnight coating at 4° C. and blocked with 3% MSD Blocking Buffer for 1 h, 4-arm PEG-2K PD-1_{ecto}, 4-arm PEG-6K PD-1_{ecto}, 6-arm PEG-3.4K PD-1_{ecto}, and 6-arm PEG-10K PD-1_{ecto} (all at 5 nM, 40 μL/well) were added into the pre-coated wells. Biotin conjugated anti-human PD-L1 antibody (clone 10F.9G2, Biolegend) was used as a control. After incubation of the plate with shaking at room temperature for 2 h and washing with MSD Wash Buffer, mouse monoclonal anti-human PD-1 antibody (Thermo Fisher Scientific) was added into the 4-arm PEG PD-1_{ecto} and 6-arm PEG PD-1_{ecto} treated wells. After additional 1 h incubation at room temperature and washing, SULFO-TAG anti-mouse IgG antibody was added and the plate was incubated for 1 h. The plate was washed and read after adding 1× Read Buffer. For control wells (clone 10F.9G2), SULFO-TAG streptavidin was added.

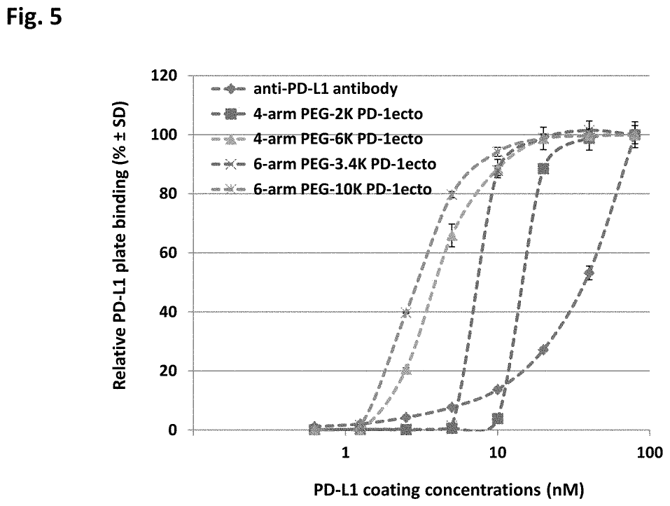

FIG. 5 shows that the pharmacophores of 4-arm PEG PD-1_{ecto} and 6-arm PEG PD-1_{ecto} exhibited strongly selective binding to high surface density of PD-L1. The discriminate binding between low and high density of PD-L1 was especially prominent for the pharmacophores with shorter linkers. As a comparison, the pharmacophores with longer linkers could also bind to the wells with lower density of PD-L1 and were less stringent in target density. The binding of anti-PD-L1 antibody to the plate was mainly in a linear mode (seen when X-axis is in normal scale).

(6) Competitive Binding Assay on PD-L1 Overexpressing SU-DHL-1 Cells.

To demonstrate the selectivity of multivalent pharmacophores to overexpressed target on cell membrane, a competitive binding assay is designed in which 4-arm PEG PD-1_{ecto} and 6-arm PEG PD-1_{ecto} compete against a PD-L1 blocking antibody for binding to PD-L1 overexpressing SU-DHL-1 cells. PC3, a low PD-L1 expressing cell is used as a control.

PE-conjugated mouse anti-human PD-L1 antibody (Clone MIN1, eBioscience) at the final concentration of 0.5 μg/100 μL (˜33.3 nM) was mixed with 4-arm PEG-2K PD-1_{ecto} or 6-arm PEG-3.4K PD-1_{ecto} titrated from 50 nM to 1 nM in Stain Buffer. SU-DHL-1 cells (0.5×10⁶ cells) were resuspended in each titration of reagent in a final volume of 100 μL per well. After incubation for 3 h on ice with occasional mixing, the cells were washed 3 times. The amount of anti-PD-L1 antibody bound to the cells was quantified by the fluorescence intensity of PE using flow cytometry.

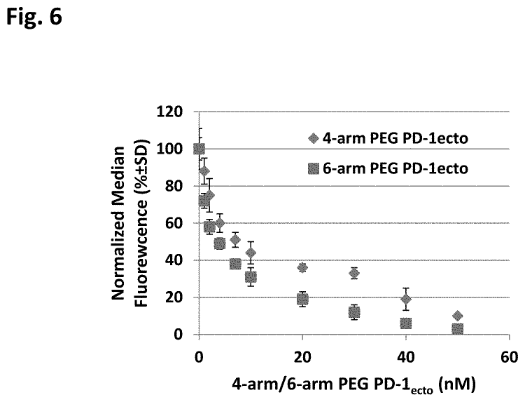

FIG. 6 shows that both 4-arm PEG-2K PD-1_{ecto} and 6-arm PEG-3.4K PD-1_{ecto} were more potent competitors for binding to SU-DHL-1 cells than the PD-L1 blocking antibody. The IC₅₀ for 4-arm PEG PD-1_{ecto} is calculated as 12.74 nM, and for 6-arm PEG-3.4K PD-1_{ecto} as 8.74 nM. Because anti-PD-L1 antibody was used at ˜33.3 nM, 4-arm PEG-2K PD-1_{ecto} is about 2.6-fold more potent than the antibody, and 6-arm PEG PD-1_{ecto} is about 3.8-fold more potent, assuming there exists a linear relationship in the competitive binding assay. The binding of 4-arm PEG PD-1_{ecto} and 6-arm PEG PD-1_{ecto} to low PD-L1 expressing PC3 cells was undetectable, and there was little competitive binding against the PD-L1 blocking antibody (data not shown).

This study demonstrates that multivalent pharmacophores exhibit overexpressed-target selectivity in cells in addition to the fixed surface targets, and have higher activity than antibody in binding to overexpressed PD-L1. Of note, the avidity of multivalent pharmacophores is also dependent on the density of the targets, and cells with different levels of PD-L1 expression will have different avidity for the same multivalent pharmacophores.

### Example 2: Overexpressed-CD47 Specific Innate Immune Checkpoint Blockade with High Avidity Tetravalent/Hexavalent PEG-SIRPα IgV-Like Domain Pharmacophores (4-Arm PEG-SIRPα IgV and 6-Arm PEG-SIRPα IgV)

### Background

Despite the tremendous success of current immune checkpoint inhibitors, including antibodies to CTLA-4, PD-1, and PD-L1, it is increasingly appreciated that these agents are efficacious for only a small population of cancer patients. The rest of patients either failed to the treatment or had short-lived responses, and some developed pronounced side effects such as serious autoimmunity. As a result, additional immune checkpoints are being explored. One intensely studied checkpoint is CD47-signal-regulatory protein-alpha (SIRP)α axis. CD47-SIRPα axis is the first and best studied innate immune checkpoint, which also include PD-1-PD-L1 axis, MHC-1-LILRB1 axis and CD24-Siglec-10 axis (Feng et al., Nat Rev Cancer, 2019, 19(10):568-86; Barkal et al., Nature, 2019, 572(7769):392-6). SIRPα is expressed on myeloid cells, including macrophages, dendritic cells and neutrophils. When bound by its ligand CD47, SIRPα becomes phosphorylated in its ITIMs (immunoreceptor tyrosine-based inhibition motifs) located in the cytoplasmic domain, which in turn recruits inhibitory molecules, in particular, protein tyrosine phosphatase SHP-1 and SHP-2, thereby preventing cell activation (Veillette and Chen, Trends Immunol, 2018, 39(3):173-84). Therefore, blockade of SIRPα-CD47 interaction could be used to promote the ability of myeloid cells, notably macrophages, to phagocytose and eliminate tumor cells. The ectodomain of SIRPα contains three Ig-like domains, a N-terminal IgV-like domain and two IgC1-like domains. The interaction between SIRPα and CD47 involves the N-terminal IgV-like domain of SIRPα and Ig-like domain of CD47 (Barclay and Van den Berg, Annu Rev Immunol, 2014, 32:25-50).

CD47, also known as integrin associated protein (IAP), is a transmembrane protein and belongs to the immunoglobulin superfamily. CD47 is expressed on virtually all cells, including red blood cells (RBCs) and platelets. One major function of CD47 is its interaction with SIRPα on myeloid cells and conveys a ‘don't eat me’ signal. Long-lived memory T cell progenitors are associated with high levels of CD47, which may support their survival by preventing clearance by macrophages. The expression of CD47 is increased in circulating hematopoietic stem cells and progenitors in order to minimize engulfment by phagocytes. CD47 is also involved in other disparate physiological processes as reviewed by Soto-Pantoja et al. (Expert Opin Ther Targets, 2013, 17(1):89-103). For example, CD47 is a high affinity receptor for extracellular matrix protein thrombospondin-1 (TSP-1), a secreted glycoprotein that plays a role in vascular development and angiogenesis. CD47 and SIRPα interaction plays a role in dendritic cell (DC) maturation, migration and antigen presentation. Mice with defects in CD47 or SIRPα show CD4 priming defect. CD47 is also involved in other physiological processes, ranging from regulation of cardiovascular homeostasis, neuronal development, bone remodeling, stem cell renewal, and cell adhesion, motility, proliferation and survival. Furthermore, it plays a key role in immune and angiogenic responses.

CD47 is overexpressed in numerous hematologic malignancies, including acute myeloid leukemia (AML), acute lymphoblastic leukemia (ALL), chronic lymphocytic leukemia (CLL), multiple myeloma (MM), myelodysplastic syndrome (MDS), and in multiple types of non-Hodgkin lymphoma (NHL), including diffuse large B-cell lymphoma (DLBCL), mantle cell lymphoma (MCL), and marginal cell lymphoma (Russ et al., Blood Rev, 2018, 32(6):480-9). Similarly, elevated CD47 expression has been demonstrated on solid tumors, including bladder, brain, breast, colon, esophageal, gastric, kidney, liver, lung, ovarian, pancreatic, and prostate cancers, and melanoma and leiomyosarcoma. CD47 has been found to be an adverse prognostic factor where high CD47 expression correlates with more aggressive disease and poorer clinical outcomes. For example, the overall survival was significantly lower for DLBCL or MCL patients who had elevated CD47 expression, and higher CD47 expression on tumor cells was associated with significantly poorer event-free survival in patients with CLL. Similar trends have been reported in other hematologic malignancies and solid tumors. In addition, there is evidence to suggest that increased CD47 expression is associated with the transition from low-risk to high-risk MDS and subsequent transformation to AML. These findings indicate that tumor cells may utilize the CD47-SIRPα pathway to evade macrophage surveillance. Blocking CD47-SIRPα axis has thus emerged as a promising therapeutic strategy. There is a large body of preclinical and emerging clinical data supporting the strategy of blocking interaction between CD47 and SIRPα in several hematological malignancies and solid tumors both as a monotherapy and as a combination treatment (Veillette and Chen, Trends Immunol, 2018, 39(3):173-84; Russ et al., Blood Rev, 2018, 32(6):480-9; Murata et al., Cancer Sci, 2018, 109(8):2349-57). CD47-SIRPα blockade is also shown to promote the development of anti-tumor adaptive T cell responses, possibly as a consequence of increased tumor cell uptake by professional antigen-presenting cells and enhanced antigen cross-presentation. However, accumulating data also suggest that SIRPα-CD47 blockade per se may not be sufficient to trigger phagocytosis of tumor cells and the engagement of prophagocytic receptors (‘eat me’ signals), such as Fc receptors, calreticulin, phosphatidylserine and SLAMF7, is also needed.

Given the nearly ubiquitous expression of CD47 at low levels in normal tissues and the homeostatic functions of the CD47-SIRPα interactions, more attention is being focused on the side effect and safety of CD47-SIRPα blockage (Veillette and Chen, Trends Immunol, 2018, 39(3):173-84; Russ et al., Blood Rev, 2018, 32(6):480-9). In fact, anemia, thrombocytopenia, and leucopenia are observed in mouse and nonhuman primate models of SIRPα-CD47 axis inhibition. The inhibition may also affect solid tissues rich in macrophages such as liver, lung, and brain. Nevertheless, research has shown that without the expression of a stress signal or prophagocytic signal, such as calreticulin, SLAMF7 or phosphatidylserine, normal cells are minimally affected. For example, some studies have revealed high levels of surface calreticulin on circulating neutrophils. Consistently, study also suggested that treatment with CD47 blocking antibody leads to depletion of neutrophils. Normal cells can upregulate calreticulin after stress, including radiation exposure and treatment with anthracycline based chemotherapy. By studying SIRPα and CD47 knockout mice, Bian et al. (Proc Natl Acad Sci, 2016, 113(37):E5434-43) discovered that, although macrophages are generally inactive toward healthy self-cells under normal condition, inflammation can trigger dramatic phagocytosis toward healthy self-cells, for which only the CD47-SIRPα blockade can restrain.

Another potential risk for CD47-SIRPα blockage is on bone formation. Osteoclast is a bone tissue-specific macrophage. CD47-induced SIRPα signaling is critical for stromal cell and osteoblasts to promote formation of osteoclasts. Blockade of the signaling impairs osteoblast differentiation, deteriorates bone formation and causes reduced formation of osteoclasts (Koshimen et al., J Biol Chem, 2013, 288(41):29333-44).

It has been reported that CD47 deficiency in tumor stromal cells increases tumor angiogenesis and tumor growth, and reduces TSP-1 expression in the tumor, leading to less macrophage recruitment to tumor (Gao et al., Oncotarget, 2017, 8(14):22406-13). Therefore, blocking CD47 on tumor stromal cells may have the potential to promote tumor progression.

Judging from all these evidence, it is clear, although little severe toxicity has been reported from clinical trials so far, non-selective blockade of CD47-SIRPα axis can potentially have various side effects, especially if used in combination with cancer chemotherapy and radiation and in case of inflammation.

Another drawback with anti-CD47 agents is so called antigen sink because of the extensive binding to CD47-expressing normal cells especially RBCs, and as a result larger and more frequent drug administration are required. To reduce the antigen sink and side effects associated with anti-CD47 treatment, one of the strategies is to use blocking antibody against SIRPα, which is expressed much more narrowly than CD47 on normal cells and thus may cause more limited toxicities (Ring et al., Proc Natl Acad Sci, 2017, 114(20):E10578-85; Ho et al., J Biol Chem, 2015, 290(20):12650-63). However, blocking antibodies against SIRPα may affect the functions of all myeloid cells that express SIRPα, including osteoclasts. Because of the sequence similarity, possible cross-reactivity to other SIRP family members may also occur, and the consequences of targeting these receptors are not yet fully understood. Moreover, the known polymorphisms in human SIRPα may render the impact of these antibodies less predictable. Bispecific antibody or fusion protein targeting CD47 and another antigen expressed on tumor cells, such as CD20, CD19, mesothelin and PD-L1, may also help in limiting bystander effects to normal cells (Piccione et al., Clin Cancer Res, 2016, 22(20):5109-19; Dheilly et al., Mol Ther, 2017, 25(2):523-33; Liu et al., Cell Rep, 2018, 24(8):2101-11). However, it will be a challenge to target CD47 with the proper affinity so that it is not too strong to bind to normal cells in a monovalent mode or too weak to efficiently block CD47 on tumor cells. In addition, the relative expression levels of both targets in the targeted tumor cells need to be considered, and a lower CD47 expression level than the other antigen may be required in order to block all CD47 expressed on the cell.

Tetravalent and hexavalent PEG-SIRPα IgV-like domain pharmacophores (4-arm PEG-SIRPα IgV and 6-arm PEG-SIRPα IgV) are compounds consisting of 4 and 6 units of SIRPα IgV-like domain which are conjugated to 4-arm or 6-arm PEG homopolymer. As described previously, these multivalent pharmacophores can have high avidity and selectivity to cancer cells overexpressing CD47. 4-arm PEG-SIRPα IgV and 6-arm PEG-SIRPα IgV can inhibit SIRPα-CD47 interactions in CD47 overexpressing cancers, while avoiding binding to normal tissues and blocking the normal immune checkpoint.

### Experimental Results:

(1) Production of SIRPα IgV-Like Domain-Mycobacterium xenopi GyrA Intein Fusion Protein and SIRPα IgV-Like Domain Hydrazide.

A method of expressed protein ligation (EPL) is used to conjugate the C-terminal IgV-like domain of SIRPα (SIRPα IgV) site-specifically to the arms of 4-arm PEG or 6-arm PEG for the production of tetravalent/hexavalent PEG-SIRPα IgV pharmacophores, similar to what is described in Example 1.

The IgV-like domain located in the N-terminus of SIRPα (SIRPα IgV) is the binding domain by CD47. The binding affinity between CD47 and SIRPα is in a range of Kd=0.3-0.5 μM (Ho et al., J Biol Chem, 2015, 290(20):12650-63). In order to determine if lower affinity of SIRPα IgV-like domain can still keep high avidity as a multivalent pharmacophore but will have lower affinity to CD47 in a monovalent binding, a Q67R mutation is introduced to the IgV-like domain and the affinity of this mutant is reduced to ˜50% of the wide-type according to Liu et al. (J Mol Biol, 2007, 365(3):680-93). Therefore, both wide-type and mutant sequence of SIRPα IgV are cloned into IMPACT vector (pTXB1, New England BioLabs) as a C-terminal fusion protein. The pTXB1 SIRPα IgV construct encodes the first 116 amino acids from SIRPα IgV-like domain with/without a Q67R mutation which is linked via leucine to the N-terminus of Mycobacterium xenopi GyrA intein that is in turn fused to the N-terminus of a chitin binding domain (CBD). The construct is transformed into E. coli strain T7 Express (New England BioLabs). A freshly grown colony is inoculated into LB medium containing 100 μg/mL ampicillin and the cells grow at 37° C. When the OD₆₀₀ of the culture reaches 0.5-0.7, protein expression is induced by adding isopropyl β-D-thiogalactopyranoside (IPTG) to a final concentration of 0.3 mM and the culture is incubated overnight at 18° C. The cells are pelleted by centrifugation at 5000 g for 20 min at 4° C., and lysed in lysis buffer (20 mM sodium phosphate pH 7.4, 0.5 M NaCl, 0.5 mM EDTA, 15% glycerol, 0.1% Sarkosyl NL) with 1 mM AEBSF by sonication. The soluble fraction is mixed with chitin beads pre-equilibrated in lysis buffer, 4° C. for 1.5 h. The beads are then washed extensively with lysis buffer followed by cleavage buffer (200 mM sodium phosphate pH 7.4, 200 mM NaCl, 0.05% Zwittergent 3-14) to yield purified SIRPα IgV GyrA intein CBD fusion protein immobilized on chitin beads. These beads are mixed with 1% hydrazine in cleavage buffer with 1 mM EDTA at room temperature overnight. The soluble supernatant contains the cleaved SIRPα IgV hydrazide, which is purified by RP HPLC on a Gilson preparative HPLC system with a Jupiter C5 column (Phenomenex) using a 60 min linear gradient, 10-100% B at a flow rate of 2 mL/min. Buffer A: 0.1% TFA/H₂O (V/V) and buffer B: 0.1% TFA/60% acetonitrile/40% H₂O (V/V/V). Fractions are analyzed by electrospray mass spectrometry and analytic RP HPLC. Pure fractions are pooled and lyophilized. The sequence of SIRPα IqV hydrazide made here is:

EEELQVIQPDKSVLVAAGETATLRCTATSLIPVGPIQ/RWFRGAGPGR
ELIYNQKEGHFPRVTTVSDLTKRNNMDFSIRIGNITPADAGTYYCVKF
RKGSPDDVEFKSGAGTELSVRA-NHNH₂

(2) Generation of Pyruvoyl 4-Arm PEG and Pyruvoyl 6-Arm PEG.

The synthesis has been described in Example 1. Two types of branched PEGs are used for linking SIRPα IgV: 4-arm PEG-2K amine, and 6-arm PEG-3.4K amine.

(3) Synthesis of 4-Arm PEG-SIRPα IgV and 6-Arm PEG-SIRPα IgV by Site-Specific C-Terminal PEGylation with Intein-Mediated Protein Ligation.

SIRPα IgV C-terminal hydrazide is dissolved in 100 μL 40% acetonitrile with 0.1% TFA to a final concentration of ˜250 μM. A 20 fold molar excess of pyruvoyl 4-arm PEG or 6-arm PEG is added and reactions left at room temperature overnight (˜16 hours).

The PEGylation reaction is diluted 10 fold in buffer A (20 mM Tris-HCl pH7.3, 0.05% Zwittergent 3-14) and loaded onto a 1 mM HiTap Q FF anion exchange column via AKTA purified system (GE Healthcare). The column is washed with 5-10 CV buffer A to remove the unbound, unreacted pyruvoyl-PEG and the bound protein is eluted over a 0.1 M NaCl gradient (20 CV). Unbound and eluted fractions are analyzed on duplicate SDS PAGE gels, one is stained with Coomassie blue and the other stained for PEG. The fractions containing the desired 4-arm PEG-SIRPα IgV and 6-arm PEG SIRPα IgV are concentrated using VivaSpin2 centrifugal concentrators (Sartorius) and run through a Superdex 200 10/300 GL column (GE Health) on 10 mM sodium phosphate pH7.4, 50 mM NaCl, 0.05% Zwittergent 3-14. This yields pure site-specifically C-terminal PEGylated multivalent SIRPα IgV pharmacophores, 4-arm PEG-SIRPα IgV and 6-arm PEG-SIRPα IgV.

(4) Competitive Binding Assay on CD47 Overexpressing Jurkat Cells.

This assay is to measure the binding avidity of multivalent PEG-SIRPα IgV pharmacophores to CD47 overexpressing Jurkat cells by competing against FITC-conjugated CD47 blocking antibody, and to compare the avidity to the affinity of wide-type SIRPα ectodomain.

FITC-conjugated mouse anti-human CD47 monoclonal antibody (Clone B6H12.2, Invitrogen) at the final concentration of 0.5 μg/100 μL (˜33.3 nM) was mixed with titrated concentration (from 0.04 nM to 10.5 μM in Stain Buffer) of SIRPα monomer (Acro Biosystems), 4-arm PEG-2K SIRPα IgV and 6-arm PEG-3.4K SIRPα IgV, both wide-type and Q67R mutant forms. The mixture was added into Jurkat cells (500,000 cells in 100 μL, final density). After incubation on ice for 3 h with occasional mixing, the cells were washed to remove the unbound reagents. The fluorescence intensity of FITC was quantified by flow cytometry. The increase of the binding activity for multivalent PEG-SIRPα IgV pharmacophores compared to SIRPα monomer can be calculated.

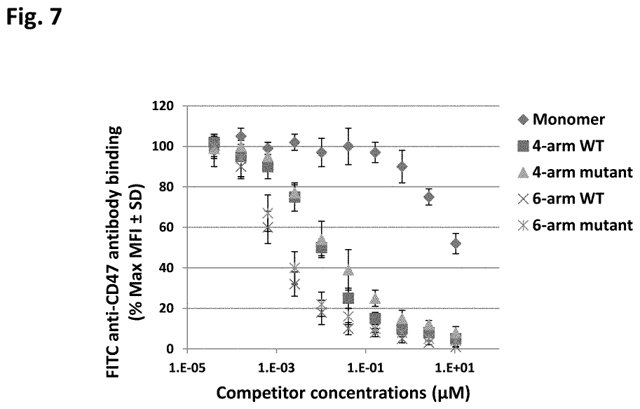

FIG. 7 shows that both 4-arm PEG-SIRPα IgV and 6-arm PEG-SIRPα IgV exhibited dramatically higher competitive binding to CD47 overexpressing Jurkat cells than wide-type SIRPα monomer. Non-linear regression analysis revealed an IC₅₀ of 8.12 nM and 0.723 nM for wide-type 4-arm PEG-SIRPα IgV and 6-arm PEG-SIRPα IgV, respectively, compared to 8.079 μM for SIRPα monomer, an increase of avidity about 995 folds and 11,174 folds, respectively. Although SIRPα Q67R mutant has about 50% reduced affinity of the wide-type SIRPα, the multivalent pharmacophores with mutant SIRPα IgV had only slightly reduced avidity with IC₅₀ of 10.91 nM and 1.06 nM for mutant 4-arm PEG-SIRPα IgV and 6-arm PEG-SIRPα IgV, respectively.

(5) Competitive Binding Assay on Human Red Blood Cells (RBCs).

To assess the risks from binding of multivalent PEG-SIRPα IgV pharmacophores to normal RBCs and subsequent anemia as has been reported for many CD47 targeting agents, a similar competitive binding assay as (4) above is conducted using RBCs as the binding targets.

Human whole blood from healthy donors was drawn into sodium heparin tube. The blood was washed 3 times with PBS and diluted in Stain Buffer (0.5×10⁶ RBCs in 100 μL, final density). The competitive binding was performed by incubating RBCs with FITC-conjugated CD47 blocking antibody (Clone B6H12.2, Invitrogen) at the final concentration of 0.5 μg/100 μL (˜33.3 nM) and titrated concentration of 4-arm, or 6-arm PEG-SIRPα IgV as described above, or an unconjugated CD47 blocking antibody (B6H12.2, Invitrogen). A mouse isotype IgG1 was used as a negative control. RBCs were incubated with the reagents on ice for 3 h, mixing occasionally. After washing, the fluorescence intensity of FITC on RBCs was quantified by flow cytometry.

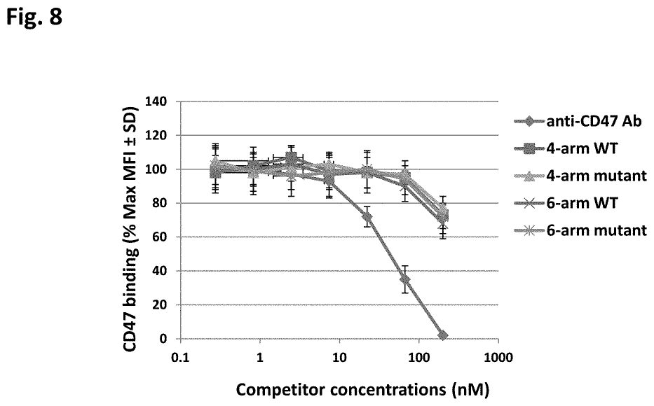

As shown in FIG. 8, 4-arm PEG-SIRPα IgV and 6-arm PEG-SIRPα IgV, either wide-type or mutant SIRPα IgV, did not compete against anti-CD47 antibody in binding to RBCs for most of the titrations except slightly at the highest dose (200 nM). In contrast, CD47 blocking antibody bound to RBCs in a dose-dependent pattern. The results from FIG. 7 and FIG. 8 together demonstrate that the multivalent PEG-SIRPα IgV pharmacophores selectively binds to CD47 overexpressing tumor cells with high avidity while avoid binding to RBCs.

(6) Hemagglutination Assay.

To further confirm that the multivalent PEG-SIRPα IgV pharmacophores do not substantially bind to RBCs and cause aggregation, a hemagglutination assay is performed.

The human RBCs were obtained as above and washed with PBS. 4 million RBCs in 100 μL PBS (final density) were plated per well in a 96-well round-bottom plate. The plate was incubated with titrated amount of 4-arm or 6-arm PEG-SIRPα IgV, anti-CD47 antibody (BRIC126, Serotec), or PBS for 4 h at 37° C. in 5% CO₂ incubator.

Hemagglutination was defined by the red or brown flocculation in the supernatant, and a lack of a significant change was defined by the clear and colorless supernatant and the sinking of RBCs to the bottom. The extent of hemagglutination was assessed by blinded scoring on a scale of 1 to 6, with 1 representing the absence of hemagglutination and 6 representing complete hemagglutination.

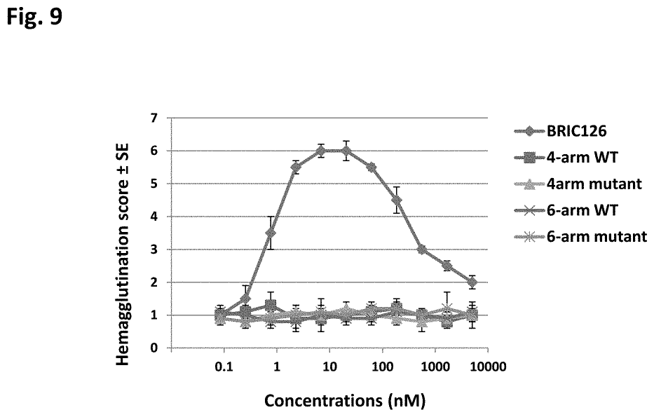

The hemagglutination assay shown in FIG. 9 indicates that, in consistent with the results of competitive binding assay on RBCs, the multivalent PEG-SIRPα IgV pharmacophores did not cause hemagglutination. In contrast, substantial hemagglutination was observed for CD47 blocking antibody (FIG. 9). The lack of significant binding of the multivalent PEG-SIRPα IgV pharmacophores to human RBCs and no hemagglutination suggest a substantial improvement of the pharmacophores over CD47 blocking antibody in selectivity and in the problem of antigen sink.

(7) In Vitro Phagocytosis Assay.

To determine if the multivalent PEG-SIRPα IgV pharmacophores can induce and enhance phagocytosis of macrophages, an in vitro phagocytosis assay was performed using human monocyte-derived macrophage to phagocytose CD47 overexpressing B cell lymphoma line Raji cells.

Preparation of human macrophages: Human peripheral blood mononuclear cells (PBMC) were prepared from blood of healthy human donors using Ficoll-Paque Plus. Monocytes were isolated by adhering PBMC to 150 mm culture plate for 1 h at 37° C. in incubator. Then, the non-adherent cells were removed by washing with PBS. The remaining cells were >95% CD14 and CD11b positive monocytes. The adherent cells were then incubated in RPMI1640 plus 10% human AB serum in the presence of 10 ng/mL human M-CSF (PeproTech) for 7-10 days to allow terminal differentiation of monocytes to macrophages. The adherent macrophages were then detached from plate using cell dissociation buffer (Sigma-Aldrich) and washed in RPMI1640 complete medium.

Raji cells were stained with 0.2 μM CellTrace CFSE (Life Technologies) and incubated with human macrophages in ultra-low attachment U-bottom 96-well plate (Corning) in the presence of 50 nM 4-arm PEG-SIRPα IgV, 6-arm PEG-SIRPα IgV, or CD47 blocking antibody B6H12.2. The plating number of macrophage to target cells was 50,000 to 250,000. For combination with tumor-opsonizing antibody, an anti-CD20 antibody rituximab was added at concentration of 0.01 μg/m L. Cells were incubated at 37° C. in 5% CO₂ incubator for 2 h. PBS and mouse IgG1 isotype were used for background phagocytosis (control).

For flow cytometric analysis of the phagocytosis, the cells were stained with near-IR Live/Dead Fixable Stain (Invitrogen), APC-conjugated anti-human CD14 (Clone 61D3, eBioscience), and PE-conjugated anti-human CD11b (Clone ICRF44, eBioscience). Cells were then washed and resuspended in Stabilizing Fixative (BD Biosciences), and the results acquired with BD FACSCalibur. The data were analyzed with FlowJo software (Tree Star, Inc.). To select single cell population, the cells were gated with FSC-H vs. FSC-A, followed by SSC-H vs. SSC-A. The phagocytosis was assessed as the percent of macrophages that are CFSE⁺ CD14⁺ CD11b⁺.

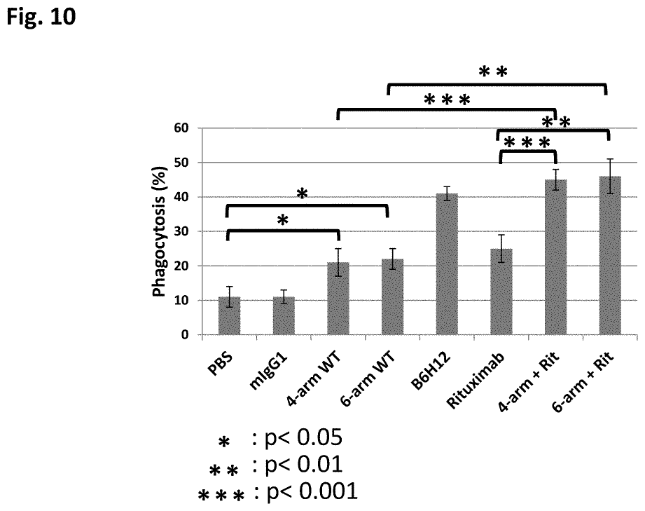

FIG. 10 shows that both multivalent PEG-SIRPα IgV pharmacophores substantially induced phagocytosis of Raji cells by macrophages. It is also clearly shown that B6H12.2, a CD47 blocking antibody was more potent than the multivalent pharmacophores in the phagocytosis, likely due to the function of Fc domain of the antibody. However, the pharmacophores could enhance the phagocytosis induced by rituximab when combined, suggesting that when pro-phagocytic signal is present, blockage of CD47-SIRPα interaction by the pharmacophores can augment the phagocytic effect.

### Example 3: Overexpressed-uPAR Specific Competitive Inhibitors—High Avidity Tetravalent/Hexavalent PEG Growth Factor-Like Domain of uPA (GFD) Pharmacophores (4-Arm PEG-GFD and 6-Arm PEG-GFD)

### Background

The urokinase-type plasminogen activator (uPA)-mediated plasminogen activation system consists of uPA, its specific receptor (uPAR, CD87) and the two inhibitors, plasminogen activator inhibitor-1 (PAI-1) and PAI-2. This system is present in the niches of bone marrow stem cells, striated muscles, and multiple types of cells including neural cells, monocytes, neutrophils, activated T cells, epithelial and endothelial cells. It is involved in the regulation of important biological processes, such as inflammation, angiogenesis, myogenesis, and neural repair (Mahmood et al., Front Oncol, 2018, 8:24; Dergilev et al., Acta Naturae, 2018, 10(4):19-32; Merino and Yepes, J Neurol Exp Neurosci, 2018, 4(2):24-29; Montuori et al., Transl Med UniSa, 2016, 15(3):15-21).

uPA is synthesized and released as a single polypeptide chain glycosylated zymogen, named pro-uPA, which consists of three domains: a N-terminal growth factor-like domain (GFD) and kringle domain (KD) and a C-terminal serine protease domain. A cleavage of the polypeptide between Lys158 and Ile159 located at the linker region produces a two-chain active form of uPA. Following another round of proteolysis at the peptide bond between Lys135 and Lys136, the two-chain form of uPA is further cleaved into two parts, a catalytically active low-molecular weight form of uPA containing the serine protease domain, and a amino-terminal fragment (ATF) that consists of GFD and KD. uPA and pro-uPA bind to uPAR through GFD. Due to the presence of GFD, ATF, pro-uPA and uPA can all bind to uPAR at a similarly high affinity with Kd<0.5 nM (Ploug et al., Biochemistry, 2001, 40(40):12157-68; Lin et al., J Biol Chem, 2010, 285(14):10982-92). Binding of uPA to uPAR dramatically enhances the efficiency of uPA catalyzed plasminogen activation (Ellis et al., J Biol Chem, 1991, 266(19):12752-8).

uPAR is a multidomain glycolipid-anchored membrane protein expressed by a variety of cells including neutrophils, T lymphocytes, monocytes, macrophages, endothelial cells and fibroblasts. By focalizing plasminogen activation to cell surface, uPAR plays an important role in extracellular matrix (ECM) remodeling, promotes tumor cell invasion, migration, and homing to distant organ. Activation of plasminogen also triggers a cascade of proteolytic events involving matrix metalloproteases (MMPs), collagenase and stromolysin-1, and leads to active degradation of ECM and activation of latent growth factors sequestered by ECM. Binding of uPA to uPAR also modulates the association of uPAR with vitronectin, G-protein-coupled chemotaxis receptors, integrins, and tyrosine kinase receptors, thereby regulating intracellular signaling and affecting angiogenesis, cell adhesion, cell migration, wound healing, inflammatory response, and cell proliferation. The chemotactic activity of uPA depends on binding to uPAR and is mediated through a chemotactic domain located in the D1-D2 linker region of uPAR, the SRSRY sequence, which interacts with formyl peptide receptor 1 (FPR1), a G protein-coupled receptor (Resnati et al., EMBO J, 1996, 15(7):1572-82; Fazioli et al., EMBO J, 1997, 16(24):7279-86; Resnati et al., Proc Natl Acad Sci, 2002, 99(3):1359-64). Specific inhibitors of the SRSRY sequence reduce the ability of cancer cells to cross Matrigel, and mesothelial and endothelial monolayers (Bifulco et al., Oncotarget, 2014, 5(12):4154-69). Binding of uPA drives uPAR into its closed conformation and enhances its affinity to vitronectin, thereby promoting lamellipodia formation and migration of the cells on vitronectin-coated matrices (Zhao et al., J Mol Biol, 2015, 427:1389-1403; Mertens et al., J Biol Chem, 2012, 287(41):34304-15; Huai et al., Nat Struct Mol Biol, 2008, 15(4):422-3). Moreover, the kringle domain of uPA is critical for the high affinity binding of uPA/uPAR complex to vitronectin (Sidenius et al., J Biol Chem, 2002, 277(31):27982-90). uPA promotes the interaction of uPAR with αvβ3 integrin and α5β1 integrin, and increases vitronectin-, fibronectin-, and laminin-dependent cell migration (Degryse et al., J Biol Chem, 2005, 280(26):24792-803). It is also reported that binding of pro-uPA to uPAR enhances the binding of cells to fibronectin through interaction between cell surface bound uPAR and α5β1 integrin (Chaurasia et al., J Biol Chem, 2006, 281(21):14852-63). Furthermore, the uPAR activated α5β1 integrin associates with and activates EGFR, which results in cell proliferation (Liu et al., Cancer Cell, 2002, 1(5):445-57). In human vascular smooth muscle cells, binding of uPA to uPAR induces its association to PDGFR-β and leads to PDGF-independent PDGFR-β activation, which elicits cell migration and proliferation (Kiyan et al., EMBO J, 2005, 24(10):1787-97). When inactive uPA:PAI-1 complex is bound to uPAR, an association between uPAR and low-density lipoprotein receptor-related protein (LRP) is initiated, and the occupied uPAR is endocytosed, which leads to regeneration of unoccupied uPAR (Czekay et al., Mol Biol Cell, 2001, 12(5):1467-79).

A large body of evidence supports that the various components of the urokinase-type plasminogen activator system, uPA, uPAR, PAI-1, and PAI-2, play major roles in tumor growth, angiogenesis, tumor cell invasion, migration, and metastasis (Mahmood et al., Front Oncol, 2018, 8:24). uPA, PAI-1 and uPAR are overexpressed in breast cancer, and the levels of uPA and PAI-1 are independent prognostic markers for poor relapse-free survival and overall survival. In prostate cancer, the expression of uPA and uPAR has a strong correlation with prostate cancer prognosis, and high levels of uPA and uPAR in the plasma correlate with increased aggressiveness, postoperative progression and metastasis. In ovarian cancer, uPAR has been reported to be overexpressed by cancer epithelial cells of 92% of ovarian cancer patients, and elevated level of soluble uPAR in the serum and urine shows association with poor survival. Elevated levels of uPA, uPAR and PAI-1 have also been reported in cervical cancer, endometrial cancer, soft-tissue sarcoma and melanoma. In colorectal cancer (CRC), uPA, uPAR and PAI-1 are highly expression in tumor, and level of soluble uPAR is elevated in plasma which is associated with poor survival. The circulated uPA and PAI-1 have been demonstrated as better prognostic markers than the commonly used colorectal cancer markers CEA and CA 19-9. Increased expression of uPA, uPAR and PAI-1 have also been found in hepatocellular carcinoma, non-small cell lung cancer, pancreatic ductal adenocarcinoma, head and neck carcinoma, and gastroesophageal cancer. Most of these cancers also show correlation between the levels of uPA-uPAR components and poor patient outcome. In acute myeloid leukemia patients, the higher expression of uPAR along with some other morphological characteristics correlates with aggressiveness of the disease and chemotherapy resistance. Ras mutations, which disables the intrinsic GTPase activity, promotes the oncogenic potential and increases the activation of PI3K/AKT and MAPK pathways, represent a common mechanism of intrinsic resistance to EGFR inhibitors in NSCLC and CRC. RAS mutations are significantly associated with higher and stronger expression of uPAR in tumor samples from NSCLC and CRC patients, and the level of uPAR is suggested to be regulated by the mutant RAS (Mauro et al., Sci Rep, 2017, 7(1):9388).

As a result, the components of the uPA-uPAR system have been identified as excellent candidates for anticancer therapies. In particular, blocking the binding of uPA to its receptor uPAR on the surface of cancer cells not only has a critical impact on pericellular proteolytic cascade but also inhibits various signaling pathways, both of which play important roles in tumor cell adhesion, invasion and metastasis. In addition, the angiogenesis in tumors may also be suppressed because of the blockage of uPAR overexpressed in endothelial cells, and decreased release of proangiogenic growth factors from ECM due to inhibition of plasminogen activation. Therefore, blocking uPA-uPAR interaction has become an attractive therapeutic strategy. It has been reported that amino-terminal fragment of uPA (ATF) can antagonize uPA-uPAR interaction, and the expression of ATF in tumor cells was shown to inhibit the invasion and metastasis of lung cancer cells (Zhu et al., DNA Cell Biol, 2001, 20(5):297-305), or decreased the invasive capacity of glioblastoma cells in vitro and made the tumor less invasive in a mouse model (Mohanam et al., Oncogen, 2002, 21(51):7824-30), or demonstrated strong anti-tumor activity in a xenograft and a mouse syngeneic breast cancer models (Li et at., Hum Gene Ther, 2005, 16(10):1157-67). The growth factor-like domain of murine uPA-Fc fusion protein is a high affinity inhibitor of interaction between mouse uPA and uPAR. It inhibited angiogenesis and tumor growth in a mouse syngeneic melanoma model (Min et al., Cancer Res, 1996, 56(10):2428-33). A linear peptide antagonist of the uPA-uPAR interaction inhibited intravasation in a chicken chorioallantoic membrane assay (Ploug et al., Biochemistry, 2001, 40(40):12157-68). The synthetic cyclic peptides, WX-360 and WX-3600Nle, competitively interfere with uPA-uPAR interaction, and reduced tumor weight and spread in a xenograft ovarian model (Sato et al., FEBS Lett, 2002, 528(1-3): 212-6). Small-molecule inhibitors have also been explored to block uPA-uPAR interaction. IPR-803 was shown binding directly to uPAR with sub-micromolar affinity, and reduced adhesion, migration and invasion of breast cancer cells in vitro and metastasis in vivo (Mai et al., Bioorg Med Chem, 2013, 21(7):2145-55). WX-UK1 is a low molecular weight serine protease inhibitor. It inhibited catalytic activity of uPA and interfered with plasminogen activation (Ertongur et al., Int J Cancer, 2004, 110(6):815-24), and inhibited cancer cell invasion. Upamostat, an orally available prodrug of WX-UK1, received FDA orphan drug designation in 2017 for the adjuvant treatment of pancreatic cancer. Blocking the engagement of uPA to uPAR can also inhibit its activity on plasminogen activation (Ellis et al., J Biol Chem, 1991, 266(19):12752-8; Vines et al., J Pept Sci, 2000, 6(9):432-9).

The development of inhibitors to uPA-uPAR system for cancer therapy has been going on for over two decades. Despite an abundance of literature demonstrating the importance of this system in the progression of many types of cancer, no uPA-uPAR targeting therapeutic agents have been developed to pass Phase II clinical trial. Besides the challenge imposed by remarkable species specificity exhibited in the uPA-uPAR interaction between human and other species, the very high affinity between uPA and uPAR (Kd<0.5 nM) makes competitive inhibitors of uPA-uPAR interaction, such as antibodies, peptides and small molecule chemicals, difficult to compete in the tumor microenvironment where both uPA and uPAR are usually overexpressed in tumors.

Multivalent pharmacophore can overcome affinity limitations when targeting uPAR overexpressing cells. In this invention, a tetravalent and hexavalent PEG-GFD pharmacophores (4-arm PEG-GFD and 6-arm PEG-GFD) are designed by conjugating units of growth factor-like domain of uPA (GFD) to 4-arm or 6-arm PEG homopolymer. As the multivalent pharmacophores, 4-arm PEG-GFD and 6-arm PEG-GFD will have sufficiently high avidity to compete against the endogenous uPA/pro-uPA in binding to the overexpressed uPAR in tumors. Moreover, due to their high specificity to overexpressed uPAR in tumors, the multivalent PEG-GFD pharmacophores will not substantially bind to normally-expressed uPAR in healthy tissues and not be interfered by soluble uPAR which is present in high concentrations in patient's blood and lymphoid circulatory systems and in tumor microenvironment. uPAR normally concentrates in cell-substratum interfaces, cell focal adhesion sites and the leading edges of the migrating tumor cells to mediate the functions of cell adhesion, migration, and invasion. The multivalent pharmacophores can cluster uPAR on the cell surface away from those locations and disrupt the associated functions.

### Experimental Results:

(1) Production of GFD-Mycobacterium xenopi GyrA Intein Fusion Protein and GFD Hydrazide.

The growth factor-like domain (GFD) located in the N-terminus of uPA is the binding domain to uPAR. The affinity between GFD and uPAR is about half of that between ATF or uPA/pro-uPA and uPAR, with Kd=0.68 nM (Ploug et al., Biochemistry, 2001, 40(40):12157-68).

A method of expressed protein ligation (EPL) is used to conjugate C-terminus of GFD site-specifically to the arms of 4-arm PEG and 6-arm PEG similar to what is described in Example 1. GFD is cloned into IMPACT vector (pTXB1, New England BioLabs) as a C-terminal fusion protein. The pTXB1 GFD construct encodes the first 43 amino acids of GFD which is linked via leucine to the N-terminus of Mycobacterium xenopi GyrA intein that is in turn fused to the N-terminus of a chitin binding domain (CBD). The construct is transformed into E. coli strain T7 Express (New England BioLabs). A freshly grown colony is inoculated into LB medium containing 100 μg/mL ampicillin and the cells grow at 37° C. When the OD₆₀₀ of the culture reaches 0.5-0.7, protein expression is induced by adding isopropyl β-D-thiogalactopyranoside (IPTG) to a final concentration of 0.3 mM and the culture is incubated overnight at 18° C. The cells are pelleted by centrifugation at 5000 g for 20 min at 4° C., and lysed in lysis buffer (20 mM sodium phosphate pH 7.4, 0.5 M NaCl, 0.5 mM EDTA, 15% glycerol, 0.1% Sarkosyl NL) with 1 mM AEBSF by sonication. The soluble fraction is mixed with chitin beads pre-equilibrated in lysis buffer, 4° C. for 1.5 h. The beads are then washed extensively with lysis buffer followed by cleavage buffer (200 mM sodium phosphate pH 7.4, 200 mM NaCl, 0.05% Zwittergent 3-14) to yield purified GFD GyrA intein CBD fusion protein immobilized on chitin beads. These beads are mixed with 1% hydrazine in cleavage buffer with 1 mM EDTA at room temperature overnight. The soluble supernatant contains the cleaved GFD hydrazide, which is purified by RP HPLC on a Gilson preparative HPLC system with a Jupiter C5 column (Phenomenex) using a 60 min linear gradient, 10-100% B at a flow rate of 2 mL/min. Buffer A: 0.1% TFA/H₂O (V/V) and buffer B: 0.1% TFA/60% acetonitrile/40% H₂O (V/V/V). Fractions are analyzed by electrospray mass spectrometry and analytic RP HPLC. Pure fractions are pooled and lyophilized. The sequence of GFD hydrazide made here is:

VPSNCDCLNGGTCVSNKYFSNIHWCNCPKKFGGQHCEIDKSKT-NHNH₂

(2) Generation of Pyruvoyl 4-Arm PEG and Pyruvoyl 6-Arm PEG.

The synthesis has been described in Example 1. Similar to what is described in Example 2, 4-arm PEG-2K-amine and 6-arm PEG-3.4K-amine are used for GFD linkage.

(3) Synthesis of 4-Arm PEG-GFD and 6-Arm PEG-GFD by Site-Specific C-Terminal PEGylation with Intein-Mediated Protein Ligation.

GFD C-terminal hydrazide is dissolved in 100 μL 40% acetonitrile with 0.1% TFA to a final concentration of ˜250 μM. A 20 fold molar excess of pyruvoyl 4-arm PEG or 6-arm PEG is added and reactions left at room temperature overnight (˜16 hours).

The PEGylation reaction is diluted 10 fold in buffer A (20 mM Tris-HCl pH7.3, 0.05% Zwittergent 3-14) and loaded onto a 1 mM HiTap Q FF anion exchange column via AKTA purified system (GE Healthcare). The column is washed with 5-10 CV buffer A to remove the unbound, unreacted pyruvoyl-PEG and the bound protein is eluted over a 0.1 M NaCl gradient (20 CV). Unbound and eluted fractions are analyzed on duplicate SDS PAGE gels, one is stained with Coomassie blue and the other stained for PEG. The fractions containing the desired 4-arm PEG-GFD and 6-arm PEG-GFD are concentrated using VivaSpin2 centrifugal concentrators (Sartorius) and run through a Superdex 200 10/300 GL column (GE Health) on 10 mM sodium phosphate pH7.4, 50 mM NaCl, 0.05% Zwittergent 3-14. This yields pure site-specifically C-terminal PEGylated multivalent PEG-GFD pharmacophores.

(4) Competitive Binding Assay on uPAR Overexpressing Hela Cells.

This assay is to measure the binding avidity of multivalent PEG-GFD pharmacophores to uPAR overexpressing Hela cells in comparison to uPA. Hela cells express uPAR but not uPA (Rabbani et al., Neoplasia, 2010, 12(10); 778-88), and therefore competition from endogenous uPA can be excluded.

Mouse anti-uPAR (Clone VIM5) is an uPAR blocking antibody (Sillaber et al., J Biol Chem, 1997, 272(12):7824-32). The potency of multivalent PEG-GFD pharmacophores and native uPA to compete against the antibody in binding to the overexpressed uPAR on the surface of Hela cells is measured and compared in this assay.

Hela cells were detached from culture flasks by trypsin/EDTA, resuspended in complete culture medium in a 50 mL conical tube, and incubated for 1 h at 37° C. in 5% CO₂ incubator to allow receptor recovery. PE-conjugated anti-uPAR antibody (Clone VIM5; Invitrogen) at the final concentration of 0.5 μg/100 μL (33.33 nM) was mixed with titrated concentrations of 4-arm PEG-GFD and 6-arm PEG-GFD, and recombinant human uPA (Biolegend) in Stain Buffer. The mixture was added into Hela cells (500,000 cells in 100 μL; final density). After incubation on ice for 3 h with occasional mixing, the cells were washed to remove the unbound test reagents. The fluorescent intensity of PE on Hela cells was quantified by flow cytometry and plotted against the titration of 4-arm PEG-GFD, 6-arm PEG-GFD and uPA. The binding avidity of multivalent PEG-GFD pharmacophores relative to native uPA can be calculated.

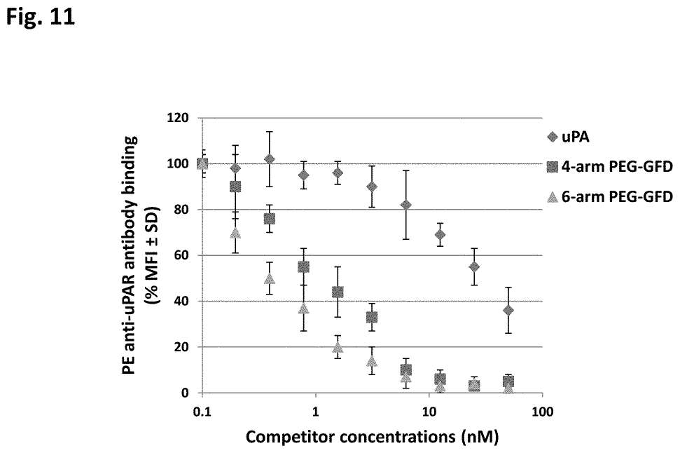

As shown in FIG. 11, 4-arm PEG-GFD and 6-arm PEG-GFD strongly competed against uPAR blocking antibody (used at ˜33.33 nM) to uPAR overexpressing Hela cells as the binding curves suggested. Non-linear regression analysis showed an IC₅₀ of 1.143 nM and 0.4117 nM for 4-arm PEG-GFD and 6-arm PEG-GFD, respectively. In contrast, the IC₅₀ for native uPA was 34.89 nM, suggesting about a 31-fold and 85-fold increase in binding activity for 4-arm and 6-arm PEG-GFD, respectively.

(5) Competitive Binding of 4-Arm and 6-Arm PEG-GFD to HT-1080 Cells in which the Overexpressed uPAR is Occupied by Endogenous uPA.

Because of the high affinity between uPA and uPAR, it is important to test if exogenously added 4-arm and 6-arm PEG-GFD can compete and dislodge the bound pro-uPA/uPA from uPAR. Human fibrosarcoma cell HT-1080 overexpresses both uPA and uPAR and the uPAR is occupied by the endogenous uPA. The potency of 4-arm PEG-GFD and 6-arm PEG-GFD to compete out uPA from uPAR was measured using this cell. As a control, the competitive potency of uPAR blocking antibody (Clone VIM5) was also tested.

HT-1080 cells were plated in 96-well plate at 30,000 cells/well in 100 μL DMEM medium supplemented with 10% FBS. After overnight incubation, the culture medium was removed and 4-arm PEG-GFD, 6-arm PEG-GFD and mouse anti-uPAR antibody (Clone VIM5, Invitrogen) with various concentrations in Stain Buffer were added into each well. The cells were incubated on ice for 0.5, 1, 2, 3 and 4 h. Then, the cells were washed twice with cold PBS and fixed in 4% paraformaldehyde for 30 min. The plate was blocked with 3% MSD Blocking Buffer for 1 h and the cells were stained with mouse anti-human uPA catalytic domain antibody (Clone 204212; R&D Systems) to quantify the amount of endogenous uPA still associated with the cells. After 2 h incubation with the antibody, HRP-conjugated anti-mouse antibody was added for another hour of incubation. TMB was then added and followed by addition of stop solution. The plate was read at 450 nm absorbance to quantify the endogenous uPA.

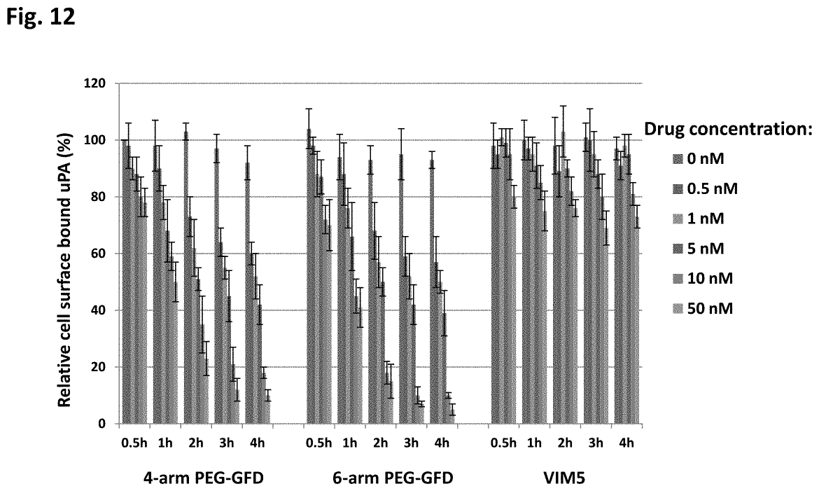

The results shown in FIG. 12 reveal that displacement of bound uPA from uPAR was time- and dose-dependent for the multivalent pharmacophores, with 3-hour incubation needed to reach close to maximal effect. Due to its higher valency, 6-arm PEG-GFD was generally more potent than 4-arm PEG-GFD when used at higher doses for 2 h and longer time incubation, when statistically analyzed. In contrast to the effective displacement of cell-bound uPA by the multivalent pharmacophores, the uPAR blocking antibody, VIM5, was much less potent. Even when used at the concentrations of 10 and 50 nM, the antibody showed limited competition against the endogenously bound uPA.

By blocking the interaction between uPA/pro-uPA and uPAR, the multivalent PEG-GFD pharmacophores will inhibit the activation of plasminogen and signaling pathways initiated by the interaction, thereby inhibiting cancer cell's adhesion, migration and invasion. To demonstrate such activities, cell adhesion, chemotaxis and Matrigel invasion assays were conducted.

(6) Cell Adhesion Assay

For adhesion of cancer cells to vitronectin, 96-well plate was coated overnight with vitronectin (Thermo Fisher Scientific) at 5 μg/mL in PBS at 4° C., 50 μL/well. The wells without coating were used as negative controls. The plate was then blocked with 3% MSD Blocking Buffer for 2 h at room temperature. Four uPAR overexpressing human cancer lines were used for the assay: non-small cell lung cancer line H1299, colorectal cancer line SW480, fibrosarcoma line HT-1080 and prostate cancer line PC3. The cells were detached from culture flasks by trypsin/EDTA, resuspended in complete culture medium in a 50 mL conical tube, and incubated for 1 h at 37° C. in 5% CO₂ incubator to allow receptor recovery. Cells were then washed with serum-free medium and resuspended in DMEM medium with 0.5% BSA. 50,000 cells in 100 μL were plated into each well with various concentrations of 4-arm and 6-arm PEG-GFD pharmacophores, and uPAR blocking antibody, VIM5. The plates were incubated at 37° C. in 5% CO₂ incubator for 3 h, and washed 4 times with PBS with 0.5% BSA. The number of adherent cells in each well was quantified by CellTiter-Glo (Promega).

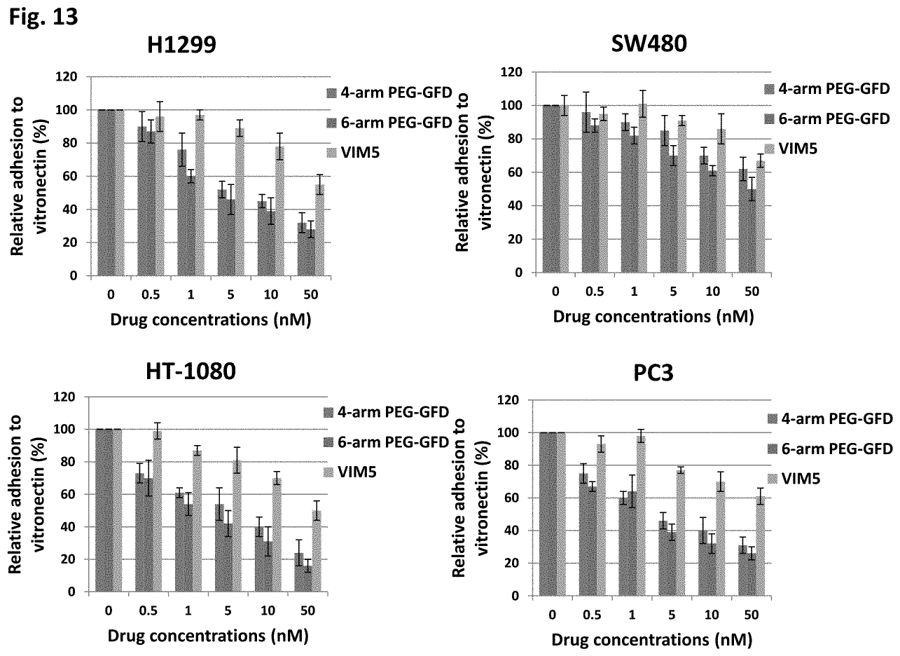

FIG. 13 shows that both 4-arm and 6-arm PEG-GFD pharmacophores inhibited the adhesion of all four cell lines to vitronectin dose-dependently, although the degrees of inhibition varied among cell lines. In consistent with what is observed for competitive binding assays, 6-arm PEG-GFD was more potent than 4-arm PEG-GFD. Again, the uPAR blocking antibody, VIM5, showed very limited inhibition of adhesion, even at the highest concentration. It's likely due to the antibody's affinity that is not high enough to dislodge the bound uPA from its receptor, uPAR.

(7) Chemotaxis Assay

Chemotaxis assay was performed using BD BioCoat 24-well transwell with 8 μm pore size filter which is coated with fibronectin (Corning). For the study, H1299 and HT-1080 cells were detached from culture flasks with trypsin/EDTA, and then the trypsin was deactivated by adding the complete medium. The cells were washed with serum-free medium twice and resuspended at the final density of 50,000 cells in 500 μL DMEM medium supplemented with 0.5% BSA. The cells were treated with multivalent pharmacophores and uPAR blocking antibody for 1 h at 37° C. in 5% CO₂ incubator. 650 μL of DMEM with 10% FBS was used as chemoattractant and added into the lower chamber. Serum-free DMEM medium was used as a negative control, and no drug treatment well was considered as 100% migration. After placing the upper chamber into the lower chamber, 500 μL cell suspensions were added to the upper chamber. 4-arm PEG-GFD, 6-arm PEG-GFD and uPAR blocking antibody, VIM5, with various concentrations were added in both upper and lower chambers. The transwell was incubated at 37° C. in 5% CO₂ incubator for 16 h. At the end of the treatment, medium in the upper chamber was aspirated and the upper side of the filter was wiped with a cotton swab to remove the cells remaining in the upper side of the filter. 100 μL of medium was removed from the lower chamber and replaced by 100 μL of CellTiter-Glo solution (Promega), and the upper chamber was placed back into the lower chamber to let the cells attached on the lower side of the filter submerged in the solution. The number of cells migrated past the filter was quantify by reading the solution in plate reader after transferring into a white 96-well plates.

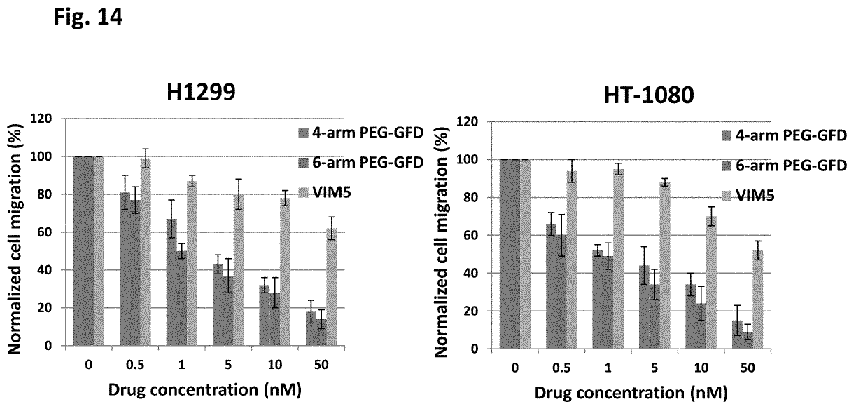

As depicted in FIG. 14, 4-arm and 6-arm PEG-GFD pharmacophores significantly reduced migration of H1299 and TH-1080 cells to the lower chamber. In contrast, uPAR blocking antibody, VIM5, was much less potent than the pharmacophores, consistent with the results of adhesion assay.

(8) Matrigel Invasion Assay

The migration of cells through Matrigel indicates the ability of the cells to digest the extracellular matrix during the process of migration. The Matrigel invasion assay is similar to the chemotaxis assay above except that the 8 μm pore size filter is coated with Matrigel. BD BioCoat Matrigel Invasion Chamber (Corning) was used for the assay with H1299 and HT-1080 cells. 4-arm PEG-GFD, 6-arm PEG-GFD and the blocking antibody VIM5 were used at the concentration of 10 nM. After 24 h treatment, the Matrigel along with the cells remaining on the upper surface of filter in the upper chamber was scraped off with a cotton swab. The migrated cells were measured with CellTiter-Glo as described above.

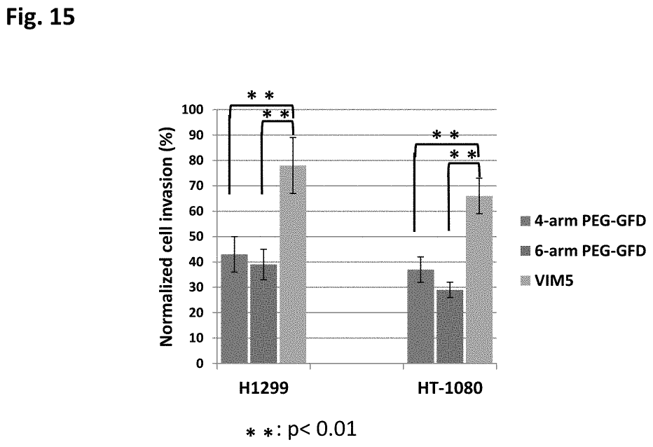

As shown in FIG. 15, both 4-arm and 6-arm PEG-GFD pharmacophores significantly inhibited the Matrigel invasion of H1299 and HT-1080 cells when treated at 10 nM. uPAR blocking antibody, VIM5, also slowed the invasion, but was much less potent than the pharmacophores.

The experiments of (6), (7) and (8) described above demonstrate, by competitive binding to overexpressed uPAR, the multivalent PEG-GFD pharmacophores substantially inhibited the adhesion, migration and invasion of cancer cells that overexpress uPAR. Their activities were much more potent than uPAR blocking antibody due to the much stronger binding avidity.

It is understood that the examples and embodiments described herein are for illustrative purposes only. Many more targets and various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

I claim:

1. A multivalent pharmacophore comprising 3 to 10 monomers or ligands linked by a branched or star-shaped linker, wherein said multivalent pharmacophore specifically binds to the overexpressed cognate targets with high avidity on the surface of cancer cells and/or non-cancer cells located in the tumor microenvironment.

2. The multivalent pharmacophore according to claim 1, wherein said monomers or ligands are the extracellular domains of membrane protein (ectodomains) that can bind to their cognate targets on cell membrane.

3. The extracellular domains of membrane protein (ectodomains) according to claim 2, which have full length sequence of said ectodomains, or are a fragment or truncated version thereof.

4. The extracellular domains of membrane protein (ectodomains) according to claim 2, which possess the native polypeptide sequences, or have one or more amino acids mutated.

5. The extracellular domains of membrane protein (ectodomains) according to claim 2, which have the same binding affinity as the native ectodomains, or have up to 5-fold higher or up to 100-fold lower binding affinity than the native ectodomains.

6. The extracellular domains of membrane protein (ectodomains) according to claim 2, which are the ectodomains of PD-1.

7. The extracellular domains of membrane protein (ectodomains) according to claim 2, which are the N-terminal IgV-like domains of signal regulatory protein-alpha (SIRPα), with or without Q67R mutation.

8. The multivalent pharmacophore according to claim 1, wherein said monomers or ligands are natural ligands that can bind to their cognate targets on cell membrane, and include, but not limit to, enzymes, zymogens, hormones, cytokines, chemokines, components of extracellular matrix, folate, and glycan containing biomolecules.

9. The natural ligands according to claim 8, which have full length sequence of the endogenous polypeptides or polysaccharides of said natural ligands, or are a fragment or truncated version thereof.

10. The natural ligands according to claim 8, which possess the endogenous polypeptide sequences of said natural ligands, or have one or more amino acids mutated.

11. The natural ligands according to claim 8, which possess the endogenous polysaccharide sequences of said natural ligands, or have one or more sugar units changed.

12. The natural ligands according to claim 8, which have the same binding affinity as the endogenous natural ligands, or have up to 5-fold higher or up to 100-fold lower binding affinity than the endogenous natural ligands.

13. The natural ligands according to claim 8, which are the N-terminal growth factor-like domains (GFD) of urokinase-type plasminogen activator (uPA).

14. The multivalent pharmacophore according to claim 1, wherein said monomers or ligands are synthetic ligands that can bind to their cognate cell surface targets and include, but not limit to, single chain variable fragments (scFv), single-domain antibodies, affimers, aptamers, peptides, cyclic peptides, D-peptides, and chemical compounds.

15. The synthetic ligands according to claim 14, which have low to moderate binding affinity to their cognate targets when measured as a monovalent interaction, where said low to moderate binding affinity is specified as dissociation constant (K_{d}) in the range of 0.01 μM and 10 μM for said monovalent interaction.

16. The overexpressed cognate targets according to claim 1, which are cell surface proteins or cell membrane-associated non-protein components that are overexpressed in cancer cells and/or non-cancer cells in the tumor microenvironment, and include, but not limit to, PD-L1, PD-L2, PD-1, B7-H3, B7x, B7-H4, galectins, TIM-3, CD74, CD47, CD24, CXCR4, folate receptor, transferrin receptor (TfR), EGFR, EGFRvIII, HER2, HER3, HER4, PDGFRα and β, FGFRs, ALK, EphA2, insulin-like growth factor receptors (IGF-1R and INSR-A), ATP-binding cassette (ABC) transporters (P-gp, BCRP and MRP1), claudins, EpCAM, carcinoembryonic antigen-related cell adhesion molecules (CEA and CEACAM6), CD44, integrins, urokinase-type plasminogen activator receptor (uPAR), type II transmembrane serine proteases (matriptase, hepsin and TMPRSS2), proteoglycans (CSPG4, glypicans and syndecans), mucins, mesothelin, carbonic anhydrase IX and XII, cancer-testis antigens (MAGEs and NY-ESO-1), and gangliosides (GD2 and GD3).

17. The multivalent pharmacophore according to claim 1, which binds to the same sites of the overexpressed cell surface targets as the endogenous ectodomains and natural ligands and acts as a competitor, or binds to non-competitive sites of the overexpressed cell surface targets.

18. The multivalent pharmacophore according to claim 1, wherein said monomers or ligands of said pharmacophore bind to the same type of targets (mono-specific), or to more than one types of targets (multi-specific).

19. The multivalent pharmacophore according to claim 1, wherein the branched or star-shaped linker has 3 to 10 branches, to which monomers or ligands are conjugated on the free end of the branches of said linker.

20. The branched or star-shaped linker according to claim 19, wherein the branches of said linker extend from one common stem or central core of the linker, such that all the linked monomers or ligands are grouped in a form of cluster and are close to each other, and the distance between the monomers or ligands is not as varied as would be with a linear linker.

21. The branched or star-shaped linker according to claim 19, wherein the branches of said linker have a length between 2 nm to 60 nm, with the specific length decided by the density of the overexpressed cognate targets and the freedom and accessibility of the linked monomers or ligands to their cognate targets.

22. The branched or star-shaped linker according to claim 19, wherein the branches and linker are flexible and are made of flexible molecules including, but not limiting to, poly(ethylene glycol) (PEG), poly(N-vinylpyrrolidone) (PVP), polyglycerol (PG), poly(N-(2-hydroxypropyl) methacrylamide) (PHPMA), polyoxazolines (POZs), polysaccharides, poly(amino acid), or the combination thereof.

23. A pharmaceutical composition comprising the multivalent pharmacophore according to claim 1 and one or more kinds selected from the group consisting of chemotherapeutic drugs, cytotoxic and cytostatic agents, radionuclides, immunologic adjuvants, immune effectors, cytokines, gene modifiers, and imaging agents, wherein these agents attach to the monomers or ligands or/and linker of said pharmacophore.

24. The multivalent pharmacophore according to claims 1 and 23, which is a therapeutic agent for cancers, a cancer diagnostic and/or prognostic agent, or a combined therapeutic and diagnostic agent.

25. The multivalent pharmacophore according to claims 1 and 23, which is a therapeutic and/or diagnostic agent for chronic viral, bacterial, or parasitic infectious diseases that overexpress cell surface targets in the diseased cells and immune cells.
